(19) United States
(12) Patent Application Publication
(10) Pub. No.: US 2015/0065443 A1
(43) Pub. Date: Mar. 5, 2015

(54) EMBELIN-BASED DELIVERY SYSTEM FOR WATER-INSOLUBLE ACTIVE AGENTS

(71) Applicant: UNIVERSITY OF PITTSBURGH - OF THE COMMONWEALTH SYSTEM OF HIGHER EDUCATION, Pittsburgh, PA (US)

(72) Inventors: Song Li, Wexford, PA (US); Xiang Gao, Pittsburgh, PA (US); Yixian Huang, Pittsburgh, PA (US); Jianqin Lu, Pittsburgh, PA (US)

(73) Assignee: University of Pittsburgh - of the Commonwealth System of Higher Educatioon, Pittsburgh, PA (US)

(21) Appl. No.: 14/391,001

(22) PCT Filed: Apr. 4, 2013
(86) PCT No.: PCT/US2013/035323

§ 371 Date: Oct. 6, 2014

Related U.S. Application Data
(60) Provisional application No. 61/621,352, filed on Apr. 6, 2012.

(51) Int. Cl. A61K 9/107; A61K 31/704; A61K 31/337; A61K 47/48; A61K 31/122

(52) U.S. Cl. CPC A61K 9/1075; A61K 47/48215; A61K 31/122; A61K 31/337; A61K 31/704
USPC 514/34; 560/171; 546/137; 514/689; 514/305; 514/449

(57) ABSTRACT

A composition having a structure of:

X-L-Y

wherein X comprises at least one embelin moiety;

L comprises a linker; and

Y comprises a hydrophilic moiety.

Also disclosed is a micelle that includes:

a core that includes at least one hydrophobic active agent and at least one embelin moiety; and

a hydrophilic zone surrounding the core and comprising at least one hydrophilic moiety.

This application claims the benefit of U.S. Provisional Application No. 61/621,352, filed Apr. 6, 2012, which is incorporated herein by reference in its entirety.

### ACKNOWLEDGMENT OF GOVERNMENT SUPPORT

This invention was made with government support under grant number CA128415 awarded by National Institutes of Health. The government has certain rights in the invention.

### BACKGROUND

One of the major barriers in drug development is that many drug candidates that demonstrate potent in vitro/in vivo activities have unfavorable biocompatibility/bioavailability properties that prevent them from advancing to preclinical and clinical stages. Formulating drugs of poor water solubility in proper carrier systems helps many potent drug candidates to reach clinical evaluation stage, and it can also improve the overall clinical performance of many existing drugs. Typically, large amounts of inactive surfactants (such as oil, polymers etc.) are used as drug carriers to formulate active ingredients into mixed micelles, emulsions, polymeric nanoparticles or liposomes for parental application. The presence of large amounts of carrier materials not only adds to the cost but also imposes additional safety issues. In addition, some formulations involve multiple steps and/or sophisticated procedures for preparation that renders it difficult for them to become clinical products. There remains an urgent need for developing simple and effective systems for formulating many poor water-soluble drugs.

Embelin (2,5-dihydroxy-3-undecyl-1,4-benzoquinone) is a naturally occurring alkyl-substituted hydroxyl benzoquinone from Embelia ribes BURM. It has been shown to possess antidiabetic, anti-inflammatory, antitumor, anticonvulsant, and hepatoprotective activities. Embelin also demonstrated excellent safety profiles in animals. Like many other compounds, embelin has poor water solubility and shows limited oral bioavailability.

### SUMMARY

Disclosed herein in one embodiment is a composition comprising a structure of:

X-L-Y
wherein X comprises at least one embelin moiety;
L comprises a linker; and
Y comprises a hydrophilic moiety.

A further embodiment disclosed herein is a micelle comprising:
a core that includes at least one hydrophobic active agent and at least one embelin moiety; and
a hydrophilic zone surrounding the core and comprising at least one hydrophilic moiety.

An additional embodiment disclosed herein is a composition comprising the above-described micelle and a continuous phase in which the micelle is solubilized.

Also disclosed herein are methods comprising administering a therapeutically effective amount of the above-described composition or micelle to a subject in need thereof.

The foregoing will become more apparent from the following detailed description, which proceeds with reference to the accompanying figures.

### BRIEF DESCRIPTION OF THE DRAWINGS

FIG. 1 shows the protocol for the synthesis of PEG-embelin conjugates.

FIG. 2 shows the ¹H NMR spectra (400 MHz) of PEG-embelin₂ in CDCl₃.

FIGS. 3A-3F show TEM images of self-assembled micelles of PEG_{3.5K}-EB₂ (FIG. 3A), PTX-loaded PEG_{3.5K}-EB₂ (FIG. 3C), and embelin-loaded PEG_{3.5K}-EB₂ (FIG. 3E); and particle size distribution of PEG_{3.5K}-EB₂ (FIG. 3B), PTX-loaded PEG_{3.5K}-EB₂ (FIG. 3D), and embelin-loaded PEG_{3.5K}-EB₂ (FIG. 3F). The spherical micelles with the diameter of around 20 nm were observed. The drug loading level was 1 mg/mL (EB, PTX) in PEG_{3.5K}-EB₂.

FIG. 4 shows the results of CMC measurements using pyrene as a fluorescence probe.

FIG. 5 shows the fluorescence microscopic images of PC-3 cells incubated with 1 μg/mL of nile red formulated in PEG_{3.5K}-EB₂ micelles for 2 h.

FIG. 6 is a table reporting the DLS analysis of the sizes of free and drug-loaded PEG_{3.5K}-EB₂ micelles.

FIG. 7 is a table reporting the physicochemical characterization of PTX-loaded PEG_{3.5K}-EB₂ micelles.

FIG. 8 is a graph depicting in vitro hemolysis assay of PEG_{3.5K}-EB₂ compared with PEI.

FIGS. 9A-9D are graphs showing the cytotoxicity of various compositions against several cell lines.

FIG. 10 is a table reporting the IC₅₀ of PTX and PTX-loaded micelles after 72 h incubation with different cancer cell lines.

FIG. 11 is a schematic representation of a PTX-loaded micelle as disclosed herein.

FIG. 12 are graphs showing the results of a mice model study.

FIG. 13 depicts a synthesis scheme for PEG_{5K}-embelin₂ and SV-119-PEG_{5K}-embelin₂.

FIG. 14 is a table reporting biophysical characterizations of free and drug-loaded PEG-embelin micelles.

FIG. 15A depicts the chemical structure of PEG_{5K}-EB₂. FIG. 15B is a graph showing the size distribution of free PEG_{5K}-EB₂ nanoparticles in PBS measured by dynamic light scanning (DLS). FIG. 15C depicts the transmission electron microscopy of PEG_{5K}-EB₂ micelles. FIG. 15D is a graph showing the critical micelle concentration (CMC) using pyrene as a fluorescence probe.

FIG. 16 is a graph showing the cumulative PTX release profile from PTX-loaded PEG_{5K}-EB₂ micelles and Taxol. DPBS (PH=7.4) containing 0.5% (w/v) Tween 80 was used as the release medium. T_{1/2} means the time needed to release half of the PTX from the formulations. Values reported are the means±SD for triplicate samples.

FIG. 17 is a graph showing the results of an in vitro hemolysis assay of PEG_{5K}-EB₂ compared with PEI. Both PEG_{5K}-EB₂ and PEI with two different concentrations (0.2, 1 mg/mL) were incubated with rat red blood cells (RBCs) for 4 h at 37° C. in an incubator shaker. The degree of RBCs lysis was measured spectrophotometrically (λ=540 nm) according to the release of hemoglobin in process. (2% Triton X-100 and DPBS were used as a positive and negative control, respectively). Values reported are the means±SD for triplicate samples

FIG. 18 are fluorescence microscope images of PC-3 cells that incubated with Nile red-loaded PEG_{5K}-EB₂ for 2 h. Cell nuclei were stained with Hoechest 33342 prior to observation.

FIGS. 19A-19C are graphs showing the cytotoxicity results of Taxol, free PEG_{5K}-EB₂, and PTX-loaded PEG_{5K}-EB₂ nanoparticles against two androgen-independent human prostate cancer cell lines DU145 and PC-3, the 4T1-2 mouse breast cancer cell line. Cells were treated for 72 h and cytotoxicity was determined by MTT assay. Values reported are the means±SD for triplicate samples

FIG. 20 are in vivo NIRF images of prostate cancer PC3-xenograft-beaing mice at 2, 24, 48 h following i.v. injection of PEG_{5K}-EB₂ micelles co-loaded with PTX and DiD.

FIG. 21A is a graph demonstrating enhanced antitumor activity of PTX formulated in PEG_{5K}-EB₂ micelles. BABL/c mice were inoculated s.c. with 4T1 cells (2×10⁵ cells/mouse). Five days later, mice received various treatments twice a week and tumor growth was monitored and plotted as relative tumor volume. P<0.01 (20 mg/kg PTX/PEG_{5K}-EB₂ vs. Taxol), P<0.02 (10 mg/kg PTX/PEG_{5K}-EB₂ vs. Taxol), P<0.05 (20 mg/kg PTX/PEG_{5K}-EB₂ VS 10 mg/kg PTX/PEG_{5K}-EB₂). N=5. FIG. 21B is a graph showing changes of body weight in mice receiving different treatments. FIG. 21C is a graph showing serum levels of transaminase in the mice treated with PTX/PEG_{5K}-EB₂ (20 mg PTX/kg) at the end of the study.

FIG. 22A is a graph demonstrating enhanced antitumor activity of PTX formulated in PEG_{5K}-EB₂ micelles. Nude mice were inoculated s.c. with PC-3 cells (2×10⁶ cells/mouse). A week later, mice received various treatments twice a week and tumor growth was monitored and plotted as relative tumor volume. P<0.005 (20 mg/kg PTX/PEG_{5K}-EB₂ vs. Taxol), P<0.01(10 mg/kg PTX/PEG_{5K}-EB₂ vs. Taxol), P<0.05 (20 mg/kg PTX/PEG_{5K}-EB₂ VS 10 mg/kg PTX/PEG_{5K}-EB₂), N=6. FIG. 22B is a graph showing changes of body weight in mice receiving different treatments.

FIG. 23 is a table showing characterization of DOX-loaded PEG_{5K}-EB₂ micelles.

FIG. 24 is a graph showing the inhibitory effect of TPGS, Embelin, PEG_{5K}-EB₂ on verapamil-stimulated P-gp ATPase activity. P-gp-Glo™ assay system (Promega, USA) was used to detect P-gp membrane ATPase activity changes using a coupled ATP-firefly luciferase assay. First, testing samples containing verapamil (50 μM) and TPGS, Embelin or PEG_{5K}-EB₂ (at final concentrations of 0, 10 and 100 μM) were added to 96-well plates and incubated with P-gp membrane for 5 min at 37° C. Then, the reaction was initiated by the addition of MgATP followed by another 40 minutes' incubation at 37° C. Afterwards, the samples were removed from 37° C. incubator and then ATP detection reagent was added in order to develop the luminescence. Signals were measured 20 minutes later on a plate reading luminometer (Victor² 1420 multilabel counter). Values are reported as the means±SD for triplicate samples. Na₃VO₄ was used as a selective inhibitor of P-gp. The changes of relative light unit (ΔRLU) were determined as follows: ΔRLU=(luminescence of Na₃VO₄-treated group)−(luminescence of the samples treated by the mixture of verapamil and Tested compound).

FIGS. 25A and 25B are graphs showing the cytotoxicity of DOX-loaded PEG_{5K}-EB₂, compared to free DOX, against the drug sensitive MCF-7 human breast cancer cell line and NCI/ADR-RES, a drug-resistant subline. Cells were treated by various DOX formulations for 72 h and then cytotoxicity was determined by MTT assay. Values reported are the means±SD for triplicate samples. In normal MCF-7 cells, DOX-loaded PEG_{5K}-EB₂ exhibited similar cell killing effects compared to DOX, no cell effect of free PEG_{5K}-EB₂ was noticed on these cells. However, for P-gp overexpressed cancer cell line-NCI/ADR-RES cells, DOX formulated in PEG_{5K}-EB₂ clearly showed better antitumor activity than that of DOX.

### DETAILED DESCRIPTION

### Terms

“Administration of” and “administering a” compound or agent should be understood to mean providing a compound or agent, a prodrug of a compound or agent, or a pharmaceutical composition as described herein. The compound, agent or composition can be administered by another person to the subject (e.g., intravenously) or it can be self-administered by the subject (e.g., tablets).

The term “alkyl” refers to a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 6 carbon atoms. Preferred alkyl groups have 1 to 4 carbon atoms. Alkyl groups may be “substituted alkyls” wherein one or more hydrogen atoms are substituted with a substituent such as halogen, cycloalkyl, alkoxy, amino, hydroxyl, aryl, alkenyl, or carboxyl. For example, a lower alkyl or (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy.

“Aryl” refers to a monovalent unsaturated aromatic carbocyclic group having a single ring (e.g., phenyl) or multiple condensed rings (e.g., naphthyl or anthryl), which can optionally be unsubstituted or substituted.

The term “amino” refers to an R-group having the structure —NH₂, which can be optionally substituted with, for example, lower alkyl groups, to yield an amino group having the general structure —NHR or —NR₂.

“Pharmaceutical compositions” are compositions that include an amount (for example, a unit dosage) of one or more of the disclosed micelles together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, Pa. (19th Edition).

The term “subject” refers to animals, including mammals (for example, humans and veterinary animals such as dogs, cats, pigs, horses, sheep, and cattle).

An “R-group” or “substituent” refers to a single atom (for example, a halogen atom) or a group of two or more atoms that are covalently bonded to each other, which are covalently bonded to an atom or atoms in a molecule to satisfy the valency requirements of the atom or atoms of the molecule, typically in place of a hydrogen atom. Examples of R-groups/substituents include alkyl groups, hydroxyl groups, alkoxy groups, acyloxy groups, mercapto groups, and aryl groups.

“Substituted” or “substitution” refer to replacement of a hydrogen atom of a molecule or an R-group with one or more additional R-groups such as halogen, alkyl, alkoxy, alkylthio, trifluoromethyl, acyloxy, hydroxy, mercapto, carboxy, aryloxy, aryl, arylalkyl, heteroaryl, amino, alkylamino, dialkylamino, morpholino, piperidino, pyrrolidin-1-yl, piperazin-1-yl, nitro, sulfato or other R-groups.

A “therapeutically effective amount” of the disclosed compositions is a dosage of the composition that is sufficient to achieve a desired therapeutic effect, such as inhibition of angiogenesis or an anti-tumor or anti-metastatic effect, or anti-inflammatory effect. For example, a therapeutically effective amount of a compound may be such that the subject receives a dosage of about 0.1 μg/kg body weight/day to about 1000 mg/kg body weight/day, for example, a dosage of about 1 μg/kg body weight/day to about 1000 μg/kg body weight/day, such as a dosage of about 5 μg/kg body weight/day to about 500 μg/kg body weight/day.

“Treatment” refers to a therapeutic intervention that ameliorates a sign or symptom of a disease or pathological condition after it has begun to develop. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. The phrase “treating a disease” is inclusive of inhibiting the full development of a disease or condition, for example, in a subject who is at risk for a disease, or who has a disease, such as cancer or a disease associated with a compromised immune system. “Preventing” a disease or condition refers to prophylactic administering a composition to a subject who does not exhibit signs of a disease or exhibits only early signs for the purpose of decreasing the risk of developing a pathology or condition, or diminishing the severity of a pathology or condition.

### Compositions

Disclosed herein are compositions that solubilize at least one hydrophobic active agent in an aqueous bulk or continuous phase. Moreover, the compositions disclosed herein may exhibit a synergistic effect from the combination of the embelin moiety and the active agent.

For example, disclosed herein are compositions and methods that can significantly improve the aqueous solubility of embelin, an embelin analog, and/or another water-insoluble drug via PEG (or other hydrophilic group) modification. PEG-derivatized embelin forms micelles in saline and is highly efficient in solubilizing other compounds including paclitaxel, camptothecin, and embelin itself. In one embodiment, preparation of PEG-derivatized embelin can be readily achieved with commercially available embelin. In another embodiment, a scheme has been developed to generate PEG-embelin conjugates via total synthesis. Furthermore, PEG-embelin conjugates of various PEG/embelin molar ratios and structural configurations can be readily synthesized.

In vitro tests have shown that paclitaxel formulated in PEG-derivatized embelin led to an improved antitumor activity against human prostate cancer cells PC3 and DU-145 over paclitaxel or embelin alone. The results disclosed herein demonstrate the potential of embelin derivatives as a new technology platform that may be useful as a simple, effective, dual-functional delivery system to facilitate in vivo applications of embelin and other poorly water-soluble drugs. PEG-derivatized embelins provide therapeutic benefits themselves in addition to serving as a carrier for other water-insoluble drugs.

In particular, embelin, identified primarily from the Embelia ribes plant, has been shown to be a natural small molecule inhibitor of X-linked inhibitor of apoptosis protein (XIAP). It is also a potent inhibitor of NF-κB activation, which makes it a potentially effective suppressor of tumor cell survival, proliferation, invasion, angiogenesis, and inflammation. However, embelin itself is insoluble in water, which makes it unsuitable for in vivo applications. Disclosed herein in one embodiment is a novel micelle system made by conjugating embelin to a hydrophilic polymer, polyethylene glycol 3,500 or 5,000 (PEG3500, PEG5000) through an aspartic acid bridge. The PEG_{3.5k}-embelin₂ conjugates readily form micelles in aqueous solutions with a CMC of 0.0205 mg/mL. Furthermore, the PEG-embelin micelles effectively solubilize paclitaxel (PTX) and embelin itself, two model hydrophobic drugs. Both drug-free and drug-loaded micelles were small in sizes (20˜30 nm) with low polydispersity indexes. In vitro cytotoxicity studies with several tumor cell lines showed that the micelles-formulated drugs were much more effective in cell killing than free drugs, suggesting a synergistic effect between carriers and delivered drugs. The results suggest that PEG-derivatized embelins may represent a novel and dual-functional carrier to facilitate the in vivo applications of poorly water-soluble anticancer drugs.

For example, disclosed herein is an effective strategy to prepare PEG-embelin conjugates through total synthesis. This synthesis has the flexibility with PEG of various lengths and different molar ratios of PEG/embelin and can be used to link recently reported embelin derivatives with improved antitumor activities (Chen el al., Design, synthesis and characterization of new embelin derivatives as potent inhibitors of X-linked inhibitor of apoptosis protein, Biorg. & Med. Chem. Letters 16 (2006) 5805-5808). The PEG-embelin conjugates self-assemble to form small-sized micelles with PTX, which allows effective delivery of drugs to solid tumors with poor vascularization. Low μM CMC and slow drug release profile suggest that PEG-embelin/PTX formulation may be more stable in blood than a paclitaxel formulation in clinic use (Ethanol/Cremorphor 1:1, Taxol®). Importantly, embelin and PEG-embelin show antitumor activities at moderate doses and synergize with other chemodrugs at low doses. Embelin has excellent safety profile and exhibits antiinflammatory and hepatoprotective activity in addition to antitumor effect. Micelle self-assembly allows convenient formation of mixed micelles of PEG-embelin and other active carrier molecules, such as α-tocopheryl succinate esterified to PEG₁₀₀₀ (TPSG), to incorporate multiple mechanisms against tumor resistance. In a further embodiment, a small molecule ligand for σ2 receptor to incorporate active targeting mechanism can be conjugated into the delivery system disclosed herein to provide a targeted micelle system.

In one embodiment disclosed herein is a composition having a structure of:

X-L-Y
wherein X comprises at least one embelin moiety;
L comprises a linker; and
Y comprises a hydrophilic moiety.
   In certain embodiments the composition is a conjugate wherein each embelin moiety is conjugated via a covalent attachment to the linker, and the linker is conjugated to the hydrophilic moiety via a covalent attachment. For example, the conjugate may have a structure of (X)ₐL-Y, wherein a is at least one 1, and more particularly is at least 2. In certain embodiments, subscript a may be 2, 4, 8, 16, 32 or 64.

The embelin moiety may be embelin or an embelin analog. Embelin has a structure of:

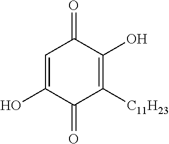

An illustrative embelin moiety has a structure of:

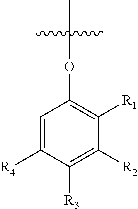

wherein each of R₁, R₃, and R₄ is individually hydroxyl, oxo, or methoxy; and R₂ is a hydrophobic moiety such as an optionally substituted alkyl having at least 6 carbon atoms, or an optionally substituted aryl, or a tautomer thereof. In particular, R₁, R₃, and R₄ are each hydroxy; and R₂ is an alkyl (particularly a straight chain alkyl) having at least 6 carbon atoms (particularly 8 to 14 carbon atoms). In certain embodiments R₂ may be an arylalkyl, an alkylaryl, or an alkyl diaryl. The embelin moiety may be coupled to the linker at any position on the embelin moiety, but preferably is coupled at the 5 position of the embelin moiety.

The linker may be any moiety that can link (preferably covalently) together the embelin moiety(s) and the hydrophilic moiety. In certain embodiments the linker can also link more than one embelin moiety (which moieties may or may not be identical structures) to a hydrophilic moiety. For example, linkers containing at least three repeating structures (which may be extended with longer spacer arms) can attach three copies of the hydrophobic carrier motif. Lysine or other suitable tri-functional molecules can be used to introduce 2, 4, 8, 16, etc., reactive groups in dendritic form which attach an equal number of copies of the embelin moiety upon conjugation. Illustrative linkers include those derived from amino acids (e.g., lysine, ornithine, aspartic acid, diaminoaryl acids (e.g., diaminobenzoic acid), diaminoalkyl acids (e.g., diaminopropyl acid), symmetrical diamino carboxylic acids), carbonyl-containing moieties (e.g., ester or carbamoyl), or ether-containing moieties. The linker may also include spacer units as described below in more detail.

In certain embodiments the linker is also cleavable (e.g., physiologically biodegradable) so that the hydrophobic carrier motif (e.g., embelin or embelin analog) and the active agent carried by the hydrophobic carrier motif are released as desired. For example, the hydrophobic carrier motif and the active agent may be released in the subject's body (which may be at a targeted tissue or organ) upon absorption or ingestion of the micelle-containing composition. Illustrative biodegradable linkers include ester, disulfide, imine, and hydrazone linkages. In other embodiments the linker is stable (i.e., non-biodegradable). Illustrative stable linkers include amide, ether, urea or carbamoyl linkages.

The hydrophilic moiety may be any group that can solubilize the composition (e.g., the micelle or the active agent-loaded micelle assembly) in an aqueous environment. The hydrophilic moiety can include an ionic or non-ionic group. Examples of anionic groups include SO₃⁻², COO⁻¹, PO₄⁻³, and the like. Examples of cationic groups include (CH₃)₃N⁺¹, (CH₃CH₂)₃N⁺¹, (HOCH₂CH₂)₃N⁺¹, methyl pyridine′, multivalent cationic groups, and the like. Amphoteric groups that include both anionic and cationic groups in the same moiety may also be utilized. Examples of non-ionic groups include polyalkylene oxides (e.g., polyethylene glycol (PEG)), polyglycerol, poly(vinyl alcohol), mono, oligo and polysaccharides and their derivatives, and the like. The hydrophilic moiety, particularly a hydrophilic polymer, may a certain molecular weight range. In the case of PEG, a MW of 300 to 20,000, more particularly 1,000 to 10,000, and most particularly 2,000 to 10,000, may be preferred.

In certain embodiments, the embelin moiety and/or the hydrophilic moiety may be functionalized so that they are activated for conjugation with the other moieties of the conjugate. For instance, the embelin moiety and/or the hydrophilic moiety may be functionalized with a dicarboxylic acid or anhydride (e.g., succinic, glutaric, citraconic or adipic) to provide appropriate reactive groups (e.g., amine, carboxyl or hydroxyl terminal groups).

In certain embodiments, bivalent, trivalent or multivalent linkers (with or without spacer units) may be provided by dendritic growth from the hydrophilic polymer moiety. In other embodiments, multivalent linkers may be provided by polyamine or polyester bridges linked directed to the hydrophilic polymer moiety. Shown below are illustrative linkage conjugation schemes:

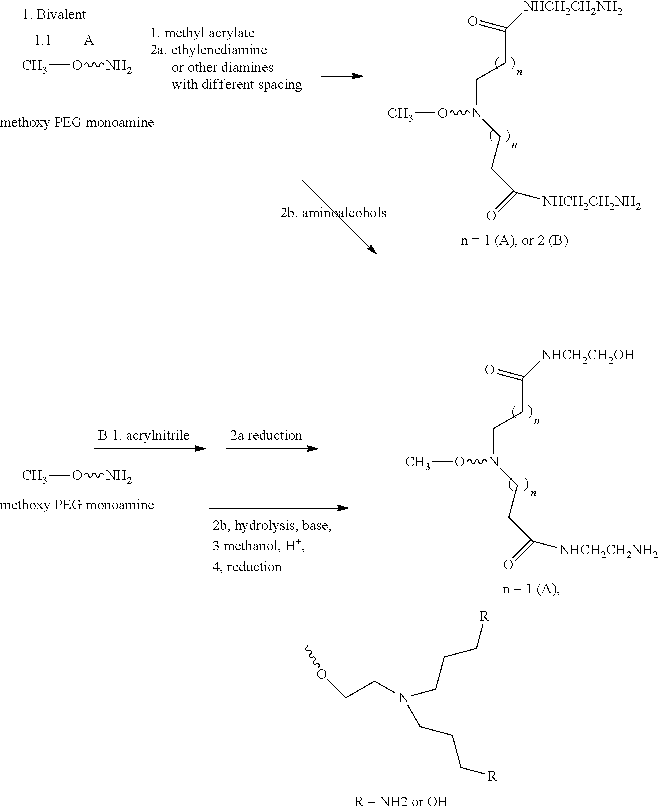

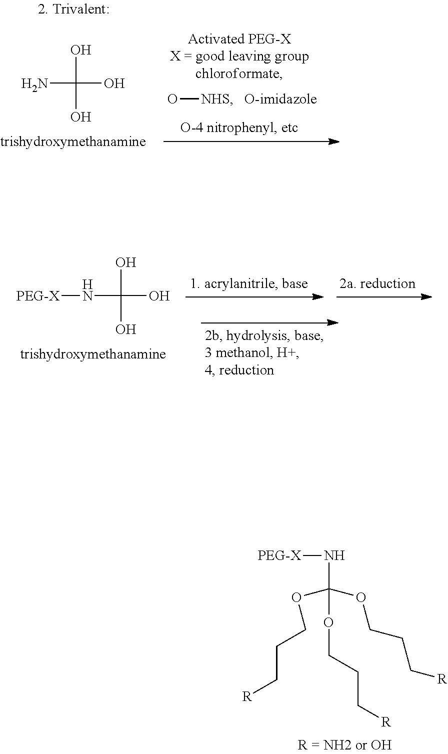

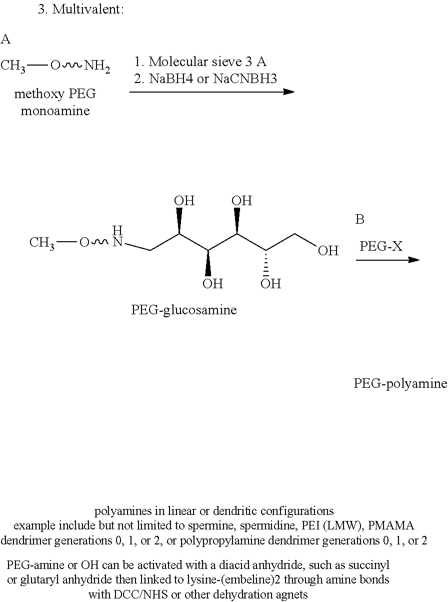

The methoxy PEG-linker intermediates shown above may be conjugated to the embelin moiety (X) at the positions shown below via functionalization activation of the linker and/or embelin moiety as described above:

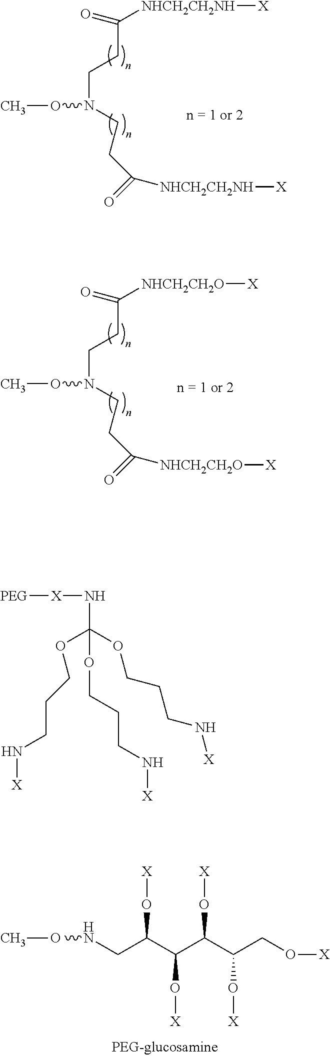

As described above, the conjugates disclosed herein form micelle systems that are solubilizers for hydrophobic active agents. FIG. 11 depicts an illustrative example of a micelle assembly. The hydrophobic active agent (e.g., PTX) resides inside a core formed by the embelin tail moieties. The embelin moieties are conjugated to the hydrophilic head moieties (e.g., PEG) via the linker (e.g., aspartic acid moiety) to provide a hydrophilic zone.

The hydrophobic active agent may be any agent that requires a carrier or some other modification to improve its solubility in an aqueous phase. Illustrative hydrophobic active agents include pharmaceutically active agents such as embelin, amphteracin B, doxurobicin, cyclosporine, FK506, taxanes (e.g. paclitaxel), and camptothecin.

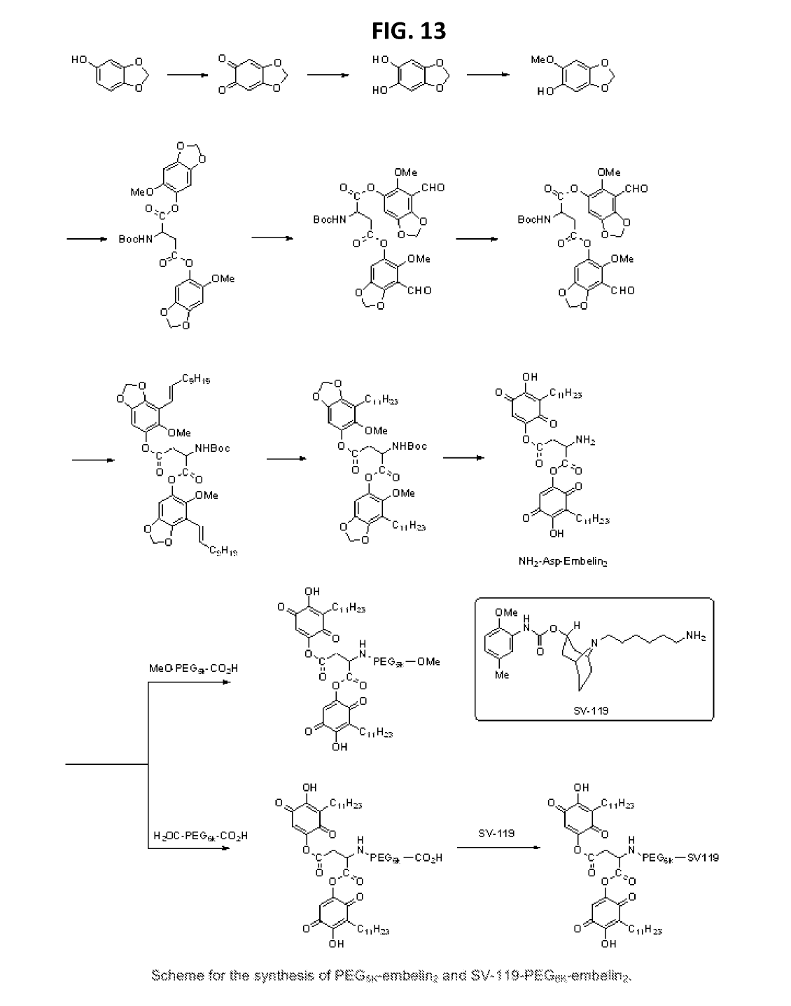

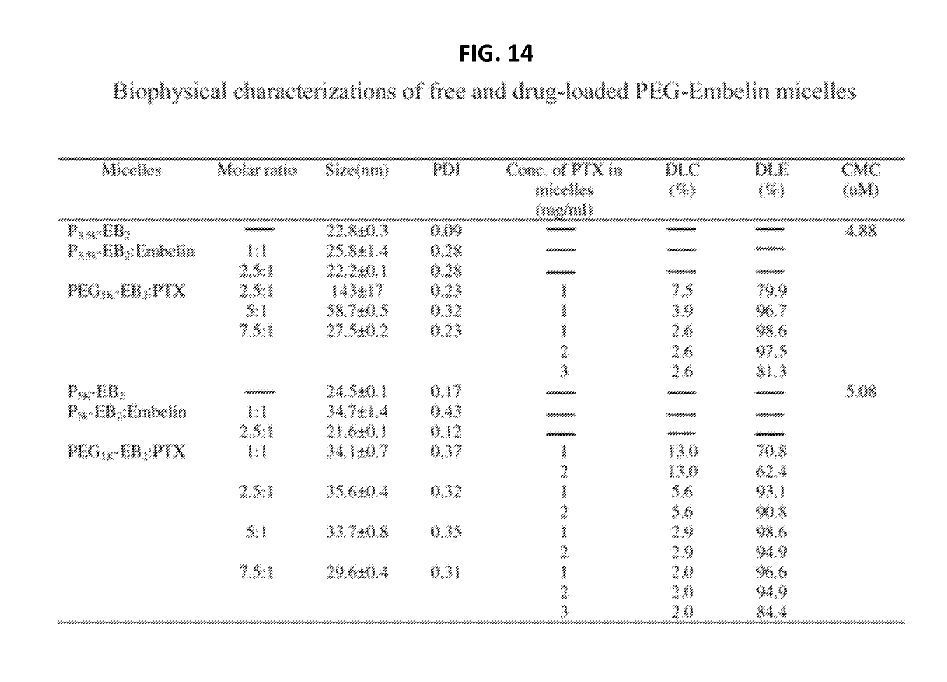

The compositions disclosed herein may also optionally include a targeting moiety. The targeting moiety assists in selectively directing the composition to a desired target such as cancer tissue. For example, σ receptors are overexpressed in a variety of human tumors including non-small cell lung carcinoma, breast cancer and PCa. There are at least three subtypes of σ receptors among which σ1 and σ2 receptors have been extensively studied. Recently σ receptor ligands have been examined as new types of anticancer agents as well as tumor-imaging agents. It has been reported that a simple σ receptor ligand, anisamide, can mediate selective delivery of liposomal doxorubicin (DOX) to PCa cells in vitro and in vivo. Recently, extensive studies demonstrated targeted delivery of siRNAs to lung cancer and melanoma using anisamide-decorated particles. In addition, cationic liposomes modified with a σ receptor ligand haloperidol have greater than 10-fold increase in transfection of MCF-7 cells. Both anisamide and haloperidol are non-selective between σ1 and σ2 subtypes. In this study we examine if 2-selective ligands can be used for targeted delivery of nanomicelles to tumor cells. The use of 2-selective ligands is preferred for tumor targeted delivery for a number of reasons. First, it was shown that there is a higher density of σ2 versus σ1 receptors in the tumor samples and various cultured tumor cells. Recent ligand binding and photoaffinity labeling studies have demonstrated lower expression levels of σ2 receptor compared with σ1 receptor in the normal tissues. Furthermore a correlation between σ2 receptor expression level and tumor proliferation state has been established both in vitro and in vivo, but such a correlation was not observed for σ1 subtype. It has been recently shown that SV-119, a σ2-specific ligand, efficiently mediated targeted delivery of liposomes to various types of cancer cells. Importantly, SV-119-decorated particles show minimal interaction with normal cells. Thus, in one embodiment disclosure herein, SV-119 may be employed to achieve active targeting of PEG-embelin nanomicelles to PCa (see FIG. 13 for a synthetic scheme for making SV-119-PEG_{6K}-embelin₂).

The solubility of the micelles or the active agent-loaded micelles in an aqueous continuous phase is greater than the very limited or non-existent solubility of the hydrophobic active agents without the micelles. In certain embodiments the micelles increase the extent of solubilization of the hydrophobic active agent (or the embelin moiety) by at least 50%, particularly at least 70%, more particularly at least 90%, and most particularly at least 95%, relative to a hydrophobic active agent-containing composition, or embelin or embelin analog-containing composition, without micelle formation. The solubilized embelin moiety and/or active agent is the amount of embelin moiety and/or active agent readily available for absorption. The micelles may be uniformly distributed in a continuous phase which may be further diluted in an aqueous medium such as water, a saline solution, simulated gastric fluid, or simulated intestinal fluid.

The critical micelle concentration (CMC) varies depending upon the identity of the particular moieties in a specific micelle assembly. However, in general, the CMC may range from 1 μM to 1 mM. In the embodiment described below in the Examples section (embelin with C11 hydrophilic side chain) the CMC is in the 1 to 10 μM range.

### Synthesis

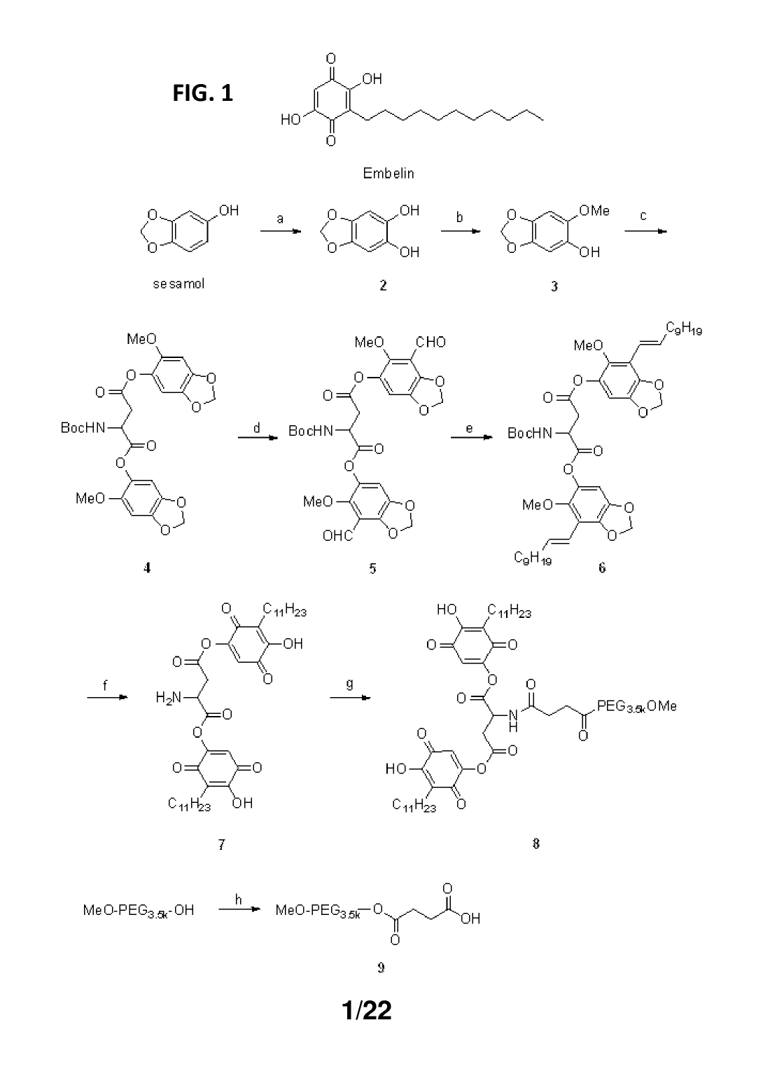

An illustrative synthesis scheme for a total synthesis of one example of a PEG-derivatized embelin conjugate is shown in FIG. 1. In certain embodiments, the molar ratio of hydrophilic moiety/embelin moiety may be from 1:1 to 1:8, more particularly 1:2 to 1:4.

In a further embodiment the hydrophilic moiety (e.g., PEG) may be directly coupled to commercially available embelin via an ester linkage. In this embodiment embelin may be derivatized with at least one succinyl anhydride group with a DMAP as a catalyst to introduce a COOH group onto the embelin moiety. The COOH group then may be activated with DCC or any other dehydration agent, with or without NHS or HObt. The activated COOH group then may be reacted amino groups of lysine which is linked to PEG through an ester bond.

An active agent-loaded micelle-containing composition may be made by mixing together the micelle carrier assembly and the active agent in an organic solvent to provide a homogeneous mixture. The removal of the organic solvent by vacuum results in a film deposit. Hydration of the film leads to formation of active agent-loaded micelles. No power input is required. In certain embodiments, the active agent loading capacity for the micelles may be at least 0.1%, more particularly at least 1%. In certain embodiments, the active agent loading capacity for the micelles may range from 1 to 10%. In certain embodiments, the nanoparticle micelle size may range from 10 to 150 nm, more particularly 20 to 35 nm.

### Administration of the Compositions

Prostate cancer (PCa) is the most commonly diagnosed malignancy, and the second leading cause of cancer mortality among American males. Although a majority of patients with metastatic PCa initially respond to chemical or surgical castration, a significant number will eventually advance to hormone-refractory PCa (HRPC), for which effective treatment is very limited. While taxanes, for example, paclitaxel (PTX) and its semi-synthetic analogues are clinically used chemo-therapeutic agents to treat various forms of cancers, the combination of docetaxel or cabazitaxel and prednisolone is the only chemotherapy regime licensed for use in HRPC. The effectiveness of PTX is limited by many factors such as inadequate formulation and side effects. Recent clinical trial data suggest that these very costly treatments only prolong a few more months of life to PCa patients over standard treatments. Therefore continuous efforts that significantly improve the outcome of the treatment are critically needed. Taxol® and Abraxane® are two FDA approved PTX formulations. Taxol is an alcohol/Cremophor formulation that is irritating and can cause hyperactivity reactions. Abraxane® is PTX-loaded human albumin nanoparticles that have a size around 130 nm, which is within the range that can penetrate well-vascularized solid tumors via an EPR effect. It is now known that for less vascularized tumors, particles with smaller size (≦64 nm) were needed for effective penetration. Thus, nanoparticles with significantly smaller sizes such as the micelles disclosed herein can offer more effective passive targeting to solid tumors, particularly poorly vascularized tumors.

Drug resistance represents another major challenge in the treatment of various types of cancers including PCa. Various mechanisms have been reported and may be differentially involved in the different stages and/or different types of cancers. In particular, alterations in the expression of components of the apoptotic machinery contribute to the chemoresistance of many tumors. X-linked inhibitor of apoptosis protein (XIAP) is the only cellular protein that has evolved to potently inhibit the enzymatic activity of mammalian caspases at both the initiation phase (caspase-9) and the execution phase (caspase-3 and -7) of apoptosis. XIAP is overexpressed in various types of cancers cells, particularly drug-resistant cancers and inhibition of XIAP has been explored as a new strategy to improve the treatment of cancers. Embelin is a naturally occurring alkyl substituted hydroxyl benzoquinone and a major constituent of Embelia ribes BURM. Embelin shows antitumor activity by itself and sensitizes cancer cells to other chemodrugs largely thorough blocking the activity of XIAP. Like many other chemodrugs, embelin is poorly water soluble. The conjugates disclosed herein form small-sized micelles (20-30 nm) and dramatically increases embelin's solubility (>200 mg/mL). More importantly, PEG-embelin becomes a highly efficient solubilizing agent for other compounds including PTX and camptothecin. The conjugates disclosed herein can serve as a safe and dual functional carrier system to achieve additive or synergistic antitumor effect with co-delivered drugs such as PTX. Indeed, the data reported below showed that delivery of PTX via PEG-embelin led to significant improvement in antitumor activity in vitro and in vivo. In addition, as described above, the delivery system may be further improved via conjugation with a small molecule ligand for σ2 receptor that is overexpressed in various types of cancers including PCa.

In certain embodiments, the disclosed compositions may be useful in the treatment of both primary and metastatic solid tumors, including carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract, (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, and meninges (including astrocytomas, gliomas, glioblastomas, retinoblastomas, neuromas, neuroblastomas, Schwannomas, and meningiomas). Such compositions may also be useful in treating solid tumors arising from hematopoietic malignancies such as leukemias (i.e. chloromas, plasmacytomas and the plaques and tumors of mycosis fungoides and cutaneous T-cell lymphoma/leukemia) as well as in the treatment of lymphomas (both Hodgkin's and non-Hodgkin's lymphomas). In addition, these compositions may be useful in the prevention of metastases from the tumors described above either when used alone or in combination with radiotherapy and/or other chemotherapeutic agents. The compounds are also useful in treating multiple myeloma.

The compositions disclosed herein can be administered systemically or locally in any manner appropriate to the treatment of a given condition, including orally, parenterally, rectally, nasally, buccally, vaginally, topically, optically, by inhalation spray, or via an implanted reservoir. The term “parenterally” as used herein includes, but is not limited to subcutaneous, intravenous, intramuscular, intrasternal, intrasynovial, intrathecal, intrahepatic, intralesional, and intracranial administration, for example, by injection or infusion. For treatment of the central nervous system, the pharmaceutical compositions may readily penetrate the blood-brain barrier when peripherally or intraventricularly administered.

In certain embodiments, the micelle-containing compositions are liquids (particularly aqueous) that are orally administered.

The dosage unit involved depends, for example, on the condition treated, nature of the formulation, nature of the condition, embodiment of the claimed pharmaceutical compositions, mode of administration, and condition and weight of the patient. Dosage levels are typically sufficient to achieve a tissue concentration at the site of action that is at least the same as a concentration that has been shown to be active in vitro, in vivo, or in tissue culture. For example, a dosage of about 0.1 μg/kg body weight/day to about 1000 mg/kg body weight/day, for example, a dosage of about 1 μg/kg body weight/day to about 1000 μg/kg body weight/day, such as a dosage of about 5 μg/kg body weight/day to about 500 μg/kg body weight/day can be useful for treatment of a particular condition.

### Examples

### PEG_{3.5K}-Embelin₂

### Experimental Procedures:

### Materials:

Paclitaxel (98%) was purchased from AK Scientific. Inc. (CA, USA). 2,5-dihydroxy-3-undecyl-1,4-benzoquinone (Embelin 98%) was purchased from 3B Scientific Corporation. (IL, USA). Dulbecco's phosphate buffered saline (DPBS) was purchased from Lonza (MD, USA). Methoxy-PEG3,500-OH, dimethyl sulfoxide (DMSO), 3-(4,5-dimethylthiazol-2-yl)-2,5-diphenyltetrazolium bromide (MTT), trypsin-EDTA solution, Nile Red, Triton X-100, Cremphor EL, and Dulbecco's Modified Eagle's Medium (DMEM) were all purchased from Sigma-Aldrich (MO, USA). Fetal bovine serum (FBS) was purchased from Life Technologies (Grand Island, N.Y.). Penicillin-streptomycin solution was from Invitrogen (NY, USA). All solvents used in this study were HPLC grade.

### Cell Culture:

DU145 and PC3 are two androgen-independent human prostate cancer cell lines. MDA-MB-231 is human breast adenocarcinoma cell line. 4T1 is mouse metastatic breast cancer cell line. All cell lines were cultured in DMEM containing 10% FBS and 1% penicillinstreptomycin in a humidified environment at 37° C. with 5% CO₂.

### Synthesis of PEG_{3.5K}-Embelin₂:

Scheme 1 (FIG. 1) shows the protocol for the synthesis of PEG-embelin conjugates. Synthesis of the intermediates and their chemical characterizations are detailed below. Compound 2: Sesamol (1.52 g, 24 mmol) in 30 mL of methanol was added to a rapidly stirred solution of Fremy's salt (7.96 g, 30 mmol) and 5.49 g (40 mmol) of KH₂PO₄ in 400 mL water at 5° C. The color of the solution changed from light brown to bright yellow within 5 min. After 30 min the quinone was extracted with 4×40 mL of ethyl acetate. The ethyl acetate solution was treated with a solution of Na₂S₂O₄ (9.0 g, 52 mmol) in water (30 mL) and after 5 min the yellow color changed to a colorless solution. The organic layer was acidified with HCl (1 N), extracted with ethyl acetate (3×30 mL), washed with water (20 mL), dried with MgSO₄, and evaporated to give 1.1 g (65%) of 2 as a light pink solid. 1H NMR ((CD₃)₂CO): δ 7.49 (s, 2H), 6.45 (s, 2H), 5.79 (s, 2H).
Compound 3: A solution of 2 (1.54 g, 10 mmol) in water (30 mL) was treated with NaOH (0.4 g, 10 mmol) while the flask was kept in an ice bath. The reaction mixture was stirred for 15 min after which MeI (1.41 g, 10 mmol) was added dropwise. The reaction mixture was then heated under reflux for 1 h, allowed to cool down to room temperature and the solvent was removed via a rotary evaporator. The crude product was purified by flash chromatography with silica gel (ethyl acetate:petroleum ether, 1:5) and pure 3 was obtained as an amber oil in 99% yield (1.68 g). ¹H NMR(CDCl₃): δ 6.11 (m, 2H), 5.88 (s, 2H), 3.72 (s, 3H).
Compound 4: A solution of N-(tert-Butoxycarbonyl)-L-aspartic acid (Boc-Asp) (2.33 g, 10 mmol) in CH₂Cl₂ (40 mL) was treated with dicyclohexylcarbodiimide (DCC) (6.2 g, 30 mmol), 4-dimethyamineopyridine (DMAP) (0.61 g, 5 mmol), and compound 3 (3.36 g, 20 mmol). The reaction vessel was capped and stirred overnight. After the reaction was completed, 100 mL Et₂O was added to the mixture. The mixture was filtered and the filtrate was concentrated under vacuum to afford the crude product. The crude product was purified by flash chromatography with silica gel (MeOH:CH₂Cl₂, 1:10) and pure 4 was obtained as an oil in 62% yield (3.31 g). ¹HNMR (CDCl3): δ 6.11 (m, 2H), 5.88 (s, 2H), 5.10 (m, 1H), 3.72 (s, 3H), 2.85 (m, 1H), 2.60 (m, 1H), 1.42 (s, 9H).
Compound 5: To a well-cooled (0-5° C.) solution of 4 (5.33 g, 10 mmol) in acetonitrile (MeCN, 10 mL), dry dimethylformamide (DMF) (0.73 g, 10 mmol) and POCl₃ (1.78 g, 11 mmol) were added with constant stirring at 0-5° C. for half an hour. The salt formed was filtered and was washed with cold MeCN. To this salt, water (20 mL) was added and heated at 50° C. for 0.5 h and then cooled. The mixture was extracted with 3×40 mL of CH₂Cl₂, washed with brine, dried over sodium sulfate, and concentrated in vacuum. The crude product was purified by flash chromatography with silica gel (MeOH:CH₂Cl₂, 1:10) and pure 5 was obtained as an oil in 80% yield (4.82 g). ¹H NMR(CDCl₃): δ 10.21 (s, 2H), 6.11 (m, 2H), 5.88 (s, 2H), 5.10 (m, 1H), 3.72 (s, 3H), 2.85 (m, 1H), 2.60 (m, 1H), 1.42 (s, 9H).
Compound 6: A solution of sodium bis(trimethylsilyl)amide (12 mL, 2 M solution in THF) was added dropwise to a stirred solution of decanyltriphenylphosphonium bromide (9.67 g, 20 mmol) in 40 mL THF, the mixture was stirred for 30 min and the solution was cooled to −78° C. To this mixture was added compound 5 (6.03 g, 10 mmol). The reaction mixture was stirred for 2 h at −78° C. and then warmed up to room temperature. The reaction mixture was quenched with saturated solution of NH₄Cl, extracted with ethyl acetate, washed with brine, dried over sodium sulfate, and concentrated in vacuum. The crude product was purified by flash chromatography with silica gel (MeOH:CH₂Cl₂, 1:10) and pure 6 was obtained as an oil in 90% yield (7.53 g). ¹H NMR (CDCl3): δ 6.11 (m, 2H), 5.88 (s, 2H), 5.10 (m, 1H), 3.72 (s, 3H), 2.85 (m, 1H), 2.60 (m, 1H), 1.42 (s, 9H). It is noted that hydrophobic groups other than —C₁₁H₂₃ could be attached to compound 5 at the positions of aldehyde groups.
Compound 7: The double bond in compound 6 was saturated by catalytic hydrogenolysis with Pd/C (10%, 500 mg) under H₂ (1 atm) in a methanol solution (8.37 g, 10 mmol in 50 mL) for 2 h. The solution was filtered to remove Pd/C followed by removal of methanol via evaporation. Into the crude product was then added 10 mL water, 10 mL MeCN, and 20 mmol CAN (ammonium ceric nitrate) (10.96 g), the solution was cooled to 0° C. and the mixture was stirred for 2 h. MeCN was removed by evaporation and the remaining solution was added to 100 mL CH₂Cl₂. The organic phase was collected and washed with brine. The CH₂Cl₂ was removed by evaporation. Then, 10 mL dioxane and 10 mL HCl were added. The mixture was stirred at room temperature for 24 h. The reaction mixture was quenched with saturated solution of NaHCO₃, extracted with ethyl acetate, washed with brine, dried over sodium sulfate, and concentrated in vacuum. The crude product was purified by flash chromatography with silica gel (MeOH:CH₂Cl₂, 1:10) and pure 7 was obtained as an oil in 42% yield (2.89 g).
Compound 8: A solution of MeO-PEG_{3.5k}-CO₂H (3.5 g, 1 mmol) in CH₂Cl₂ (5 mL) was treated with DCC (0.41 g, 2 mmol), DMAP (0.12 g, 1 mmol), and compound 7 (689 mg, 1 mmol). The reaction vessel was capped and stirred overnight. After the reaction was completed, 100 mL Et₂O was added and the mixture was filtered to afford the crude product. The crude product was purified by flash chromatography with silica gel (MeOH:CH₂Cl₂, 1:10) and pure 8 was obtained as a wax solid in 50% yield (2.1 g).

### Formation of Micelles:

Paclitaxel (PTX)- or embelin (EB)-solubilized micelles were prepared by the following method. PTX or EB (10 mM in chloroform) was added to PEG_{3.5K}-EB₂ (10 mM in chloroform) with various carrier/drug ratios. The organic solvent was first removed by nitrogen flow to form a thin film of drug/carrier mixture. The film was further dried under high vacuum for 2 h to remove any traces of remaining solvent. Drug-loaded micelles were formed by suspending the film in DPBS. The drug-free micelles were similarly prepared as described above.

### Measurement of Size and Zeta Potential:

Dynamic light scattering (DLS) (Zetasizer Nano ZS instrument, Malvern, Worcedtershire, UK) was used to measure the particle size and zeta potential of drug-free and drug-loaded micelles. Micelles were stored at 4° C., and the samples were tested for the changes in particle size and size distribution.

### Determination of PTX Loading Efficiency:

PTX-solubilized micelles were prepared at an input PTX concentration of 1.07, 2.14, and 3.21 mg/mL respectively. Aliquots of samples were filtered through 0.45 m PVDF syringe filter. PTX in the filtered and non-filtered micelles was extracted using methanol and measured by high performance liquid chromatography (HPLC, Waters). A reverse phase column (C18) was employed. The detection was performed by using UV detector at 227 nm, 70% methanol as a mobile phase, flow rate at 1.0 mL/min. Drug loading capacity (DLC) and drug loading efficiency (DLE) were calculated according to the following formula:

DLC (%)=[weight of drug used/(weight of polymer+drug used)]×100%

DLE (%)=(weight of loaded drug/weight of input drug)×100%

### Determination of the Critical Micelle Concentration (CMC):

The CMC of PEG_{3.5K}-EB₂ was determined by employing pyrene as a fluorescence probe. A drug-free micelle solution in DPBS (2.5 mg/mL) was prepared via solvent evaporation method. A series of 2-fold dilutions was then made with PEG_{3.5K}-EB₂ concentrations ranging from 7.63×10-5 to 2.5 mg/mL. At the same time, aliquots of 50 L of 4.8×10⁻⁶ M pyrene in chloroform were added into 15 separate vials. The chloroform was first removed by nitrogen flow to form a thin film. The film was further dried under high vacuum for 2 h to remove any traces of remaining solvent. Then, the pre-prepared micelle solutions (400 L in DPBS) of varying PEG3.5K-EB2 concentrations were added to the pyrene film to obtain a final pyrene concentration of 6×10⁻⁷ M for each vial. The solutions were kept on a shaker at 37° C. for 24 h to reach equilibrium before fluorescence measurement. The fluorescence intensity of samples was measured at the excitation wavelength of 334 nm and emission wavelength of 390 nm by Synergy H1 Hybrid Multi-Mode Microplate Reader (Winooski, Vt.). The CMC is determined from the threshold concentration, where the sharp increase in pyrene fluorescence intensity is observed.

### Transmission Electron Microscope (TEM):

The morphology of micelles was observed on a Jeol 1011 transmission electron microscope (TEM). The aqueous micelle solution (1.0 mg/mL) was added onto copper grids coated with Formvar, and then stained with 1% uranyl acetate. The sample processing and imaging was performed at room temperature.

### Cellular Uptake of PEG3.5K-EB2 Micelle:

The uptake study of the system was conducted via fluorescence microscopic examination with nile red as a fluorescence probe. The stock solution of nile red was prepared in acetone and stored in −20° C. The nile red-loaded micelles were prepared by using the method described above. Following the preparation nile red/PEG3.5K-EB2 thin film, DMEM was added to the flask to form the nile red-loaded micelle. PC-3 cells (15,000 cells/well) were seeded in 48-well plate in 200 L of DMEM for 24 h. Then the medium was removed and 200 L of the nile red-loaded PEG3.5K-EB2 in DMEM was added to each well. The cells were cultured at 37° C. for 2 h. Cells were washed with DPBS three times and then fixed with 4% paraformaldehyde for another 30 min at room temperature. Finally, the cells were stained with Hoechst33342 for 5 min prior to the fluorescence examination.

### Hemolysis Assay:

Fresh blood samples were collected through cardiac puncture from rats. 10 mL blood was added with EDTA-Na2 immediately to prevent coagulation. Red blood cells (RBCs) were separated from plasma by centrifugation at 1500 rpm for 10 min at 4° C. The RBCs were washed three times with 30 mL ice cold DPBS. RBCs were then diluted to 2% w/v with ice cold DPBS and utilized immediately for the hemolysis assay. One mL of diluted RBC suspension was treated with various concentrations (0.2 and 1.0 mg/mL) of PEG3.5k-EB2 and PEI, respectively, and then incubated at 37° C. in an incubator shaker for 4 h. The samples were centrifuged at 1500 rpm for 10 min at 4° C., and 100 L of supernatant from each sample was transferred into a 96-well plate. The release of hemoglobin was determined by the absorbance at 540 nm using a microplate reader. RBCs treated with Triton X-100 (2%) and DPBS were considered as the positive and negative controls, respectively. Hemoglobin release was calculated as (ODsample-ODnegative control)/(ODpositive control-ODnegative control)×100%

### In Vitro Cell Cytotoxicity:

DU145 (2000 cells/well), PC-3 (5000 cells/well), MDA-MB-231 (2000 cells/well), 4T1 (1000 cells/well) or A549 cells (2500 cells/well) were seeded in 96-well plates followed by 24 h of incubation in DMEM with 10% FBS and 1% streptomycin-penicillin. Then various concentrations of PTX (free or formulated in PEG3.5K-EB2 micelles) were added in quadruplicate and cells were incubated for 72 h. Twenty L of 3-(4,5-dimethylthiazol-2-yl)-2,5-diphenyltetrazoliumbromide (MTT) in PBS (5 mg/mL) was added and cells were further incubated for 4 h. The medium in the plates was removed and MTT formazan was solubilized by DMSO. The absorbance was measured by microplate reader with wavelength at 550 nm and reference wavelength at 630 nm. Untreated groups were used as controls. Cell viability was calculated as [(ODtreat-ODblank)/(ODcontrol-ODblank)×100%].

### Results:

### Synthesis of PEG_{3.5K}-EB₂ Conjugates:

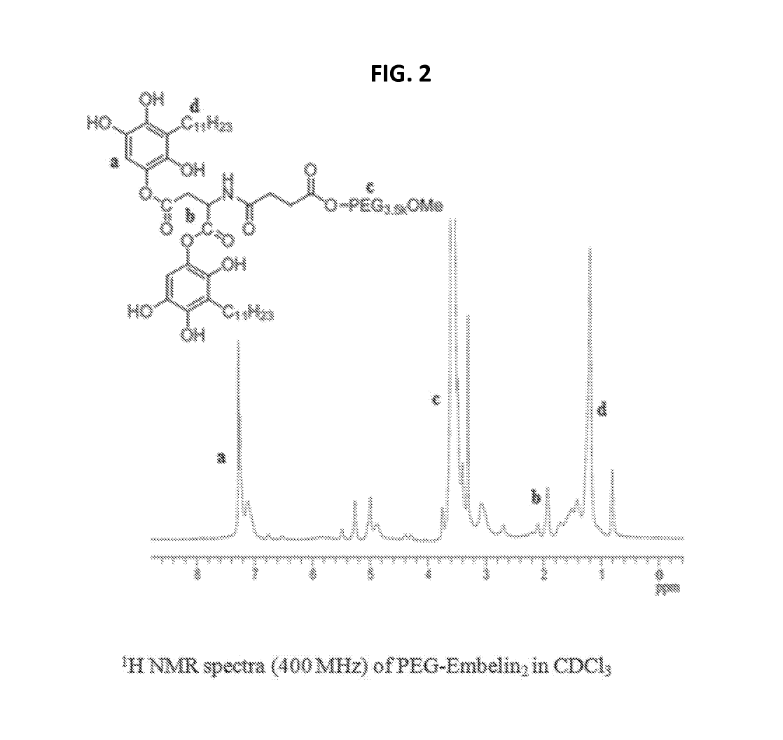

We have developed a strategy to synthesize PEG_{3.5K}-EB₂ conjugates in which two molecules of embelin were coupled to one molecule of PEG via a linker of aspartic acid. This involves the synthesis of benzoquinone followed by coupling to carboxyl groups of aspartic acid. Undecyl side chains were then installed onto each of the two benzoquinone rings. Finally PEG was coupled to aspartic acid-EB2 through the deprotected amino group. ¹H NMR spectrum of PEG-embelin₂ showed signals at 3.63 ppm attributable to the methylene protons of PEG, the embelin proton signals at 7.30 and 6.50 ppm and the carbon chain singles at 1.05-1.35 ppm. The aspartate signals were identified at 2.04 ppm (FIG. 2). The molecular weight of the PEG-embelin₂ conjugate from MALDI-TOF MS (4200) is very similar to the theoretical value (4203). These results suggest successful synthesis of PEG-embelin₂ conjugate.

### Biophysical Characterization of Micelles:

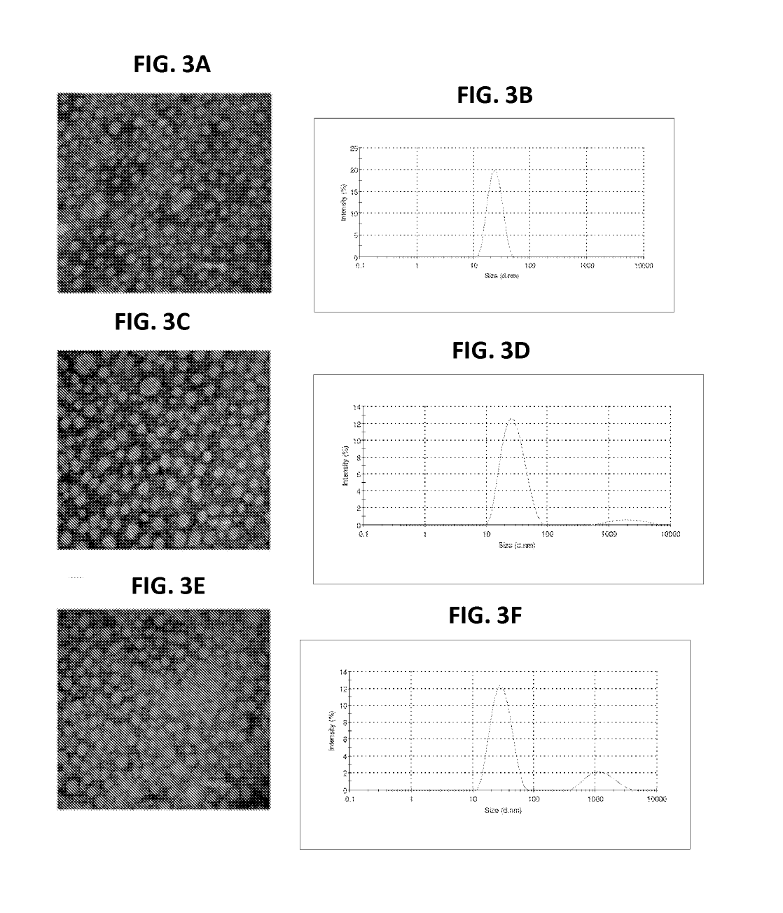

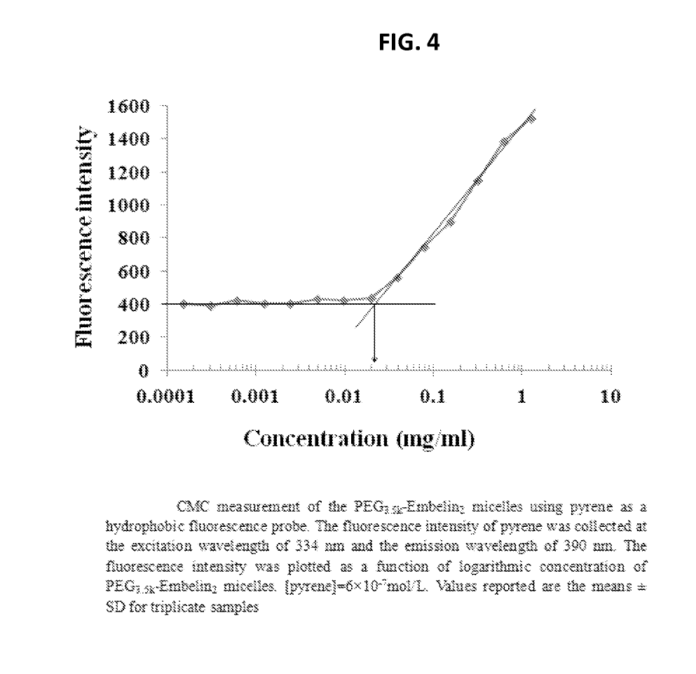

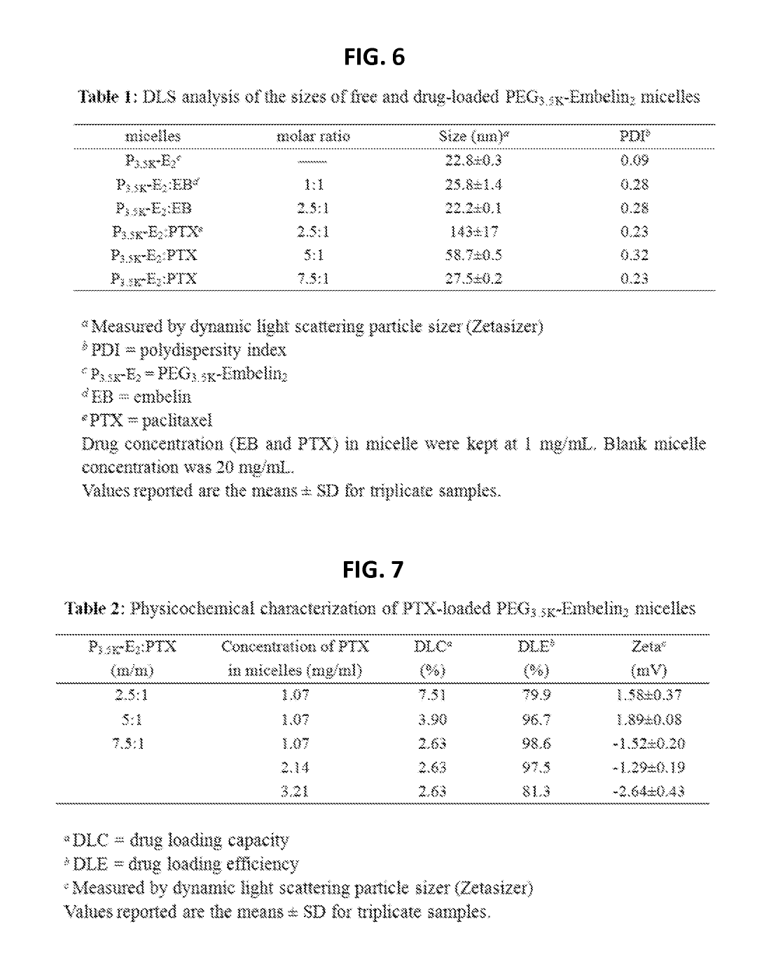

Micelles were readily prepared from PEG_{3.5K}-EB₂ conjugates via solvent evaporation method. PEG_{3.5K}-EB₂ conjugates can be dissolved in water at concentrations up to 200 mg/mL. Dynamic light scattering (DLS) measurements showed that these micelles had hydrodynamic sizes around 22 nm at the concentration of 20 mg/mL (FIG. 3A), which shall ensure efficient passive targeting to the solid tumors. FIG. 4 shows the results of CMC measurements using pyrene as a fluorescence probe. Upon incorporation into the micelles, the fluorescence intensity of pyrene increases substantially at the concentration of micelles above the CMC. Based on the partition of the pyrene, the CMC of PEG_{3.5K}-EB₂ could be obtained by plotting the fluorescence intensity versus logarithm concentration of the polymer. The CMC of PEG3.5K-EB2 was determined from the crossover point at the low concentration range. The CMC of the PEG3.5K-EB2 conjugates is 4.9 M, which is similar to most reported micellar delivery systems. PTX, a potent hydrophobic anticancer agent, was readily loaded into PEG3.5K-EB2 micelles. In addition, PEG3.5K-EB2 micelles can effectively solubilize embelin itself. FIG. 6 shows the DLS size measurement of PTX- and embelin-loaded PEG3.5K-EB2 micelles at a drug concentration of 1 mg/mL. There were little changes in sizes when PTX and embelin were loaded into micelles at a carrier/drug ratio of 7.5/1 and 2.5/1 (m/m), respectively. FIGS. 3A, 3C and 3E show the TEM images of drug-free and drug-loaded micelles after staining with 1% uranyl acetate. Spherical particles of uniform size were observed for both drug-free and drug (PTX or embelin)-loaded micelles. The sizes of the micelles observed under TEM are consistent with those measured by DLS. The size range may enable these drug-loaded micelles to take full advantage of the EPR effect and accumulate at tumor sites. FIG. 6 (Table 1) shows the sizes of PTX- or embelin-loaded micelles at different carrier/drug molar ratios. Embelin can be effectively solubilized in PEG3.5K-EB2 micelles at a carrier/drug ratio as low as 1:1 (m/m) while still maintaining the small size (˜25.8 nm) for at least 1 week. PTX-loaded PEG3.5K-EB2 micelles had relatively large size (˜143 nm) at a carrier/drug ratio of 2.5:1 (m/m) and the particles were stable for less than 1 day. Increasing the input molar ratio of PEG3.5KEB2/PTX led to gradual decrease in the size of PTX-loaded micelles. At the molar ratio of 7.5/1, the size of the PTX-loaded micelles was similar to that of drug-free micelles.

### Drug Loading Efficiency (DLE):

DLE of paclitaxel-loaded micelles was determined by HPLC and the results are shown in FIG. 7 (Table 2). DLE was as high as 79.89% when PTX was formulated in PEG3.5K-EB2 micelles at a carrier/PTX input ratio of 2.5/1 (m/m) and PTX concentration of 1.07 mg/mL. Increasing the carrier/PTX input ratios led to further increase in the drug loading efficiency. PEG3.5K-EB2/PTX formed the most stable particles at a carrier/drug ratio of 7.5/1. At this ratio, PTX was quantitatively formulated in the PEG3.5K-EB2 micelles when the PTX concentration was less than 2.14 mg/mL. Increasing the PTX concentration to 3.21 mg/mL led to a slight decrease in DLE (81.3%). The surface charges of PTX-loaded PEG3.5K-EB2 micelles were close to neutral (+1.89˜−2.64) for all particles examined.

### Intracellular Delivery of Fluorescence Dye Via PEG3.5K-EB2 Micelles:

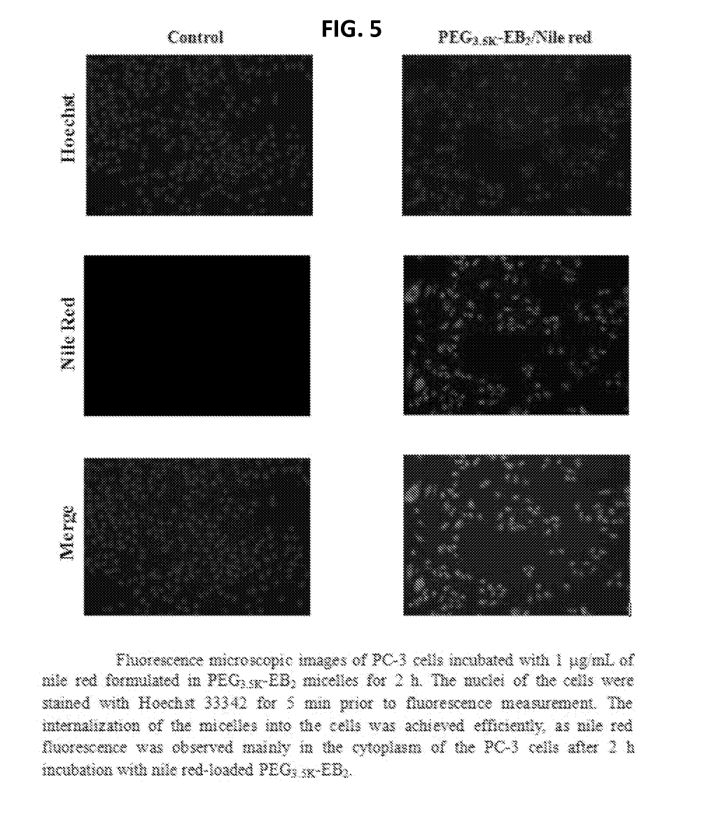

FIG. 5 shows the fluorescence images of PC3 cells 2 h following treatment of nile red formulated in PEG3.5K-EB2 micelles. Fluorescence was observed both on the cell membrane and inside of cells. Both perinuclear punctuate and diffuse distribution was observed, suggesting that nile red-loaded PEG3.5K-EB2 was largely taken up by cells via endocytosis and partially released into cytoplasm.

### Hemolysis Study:

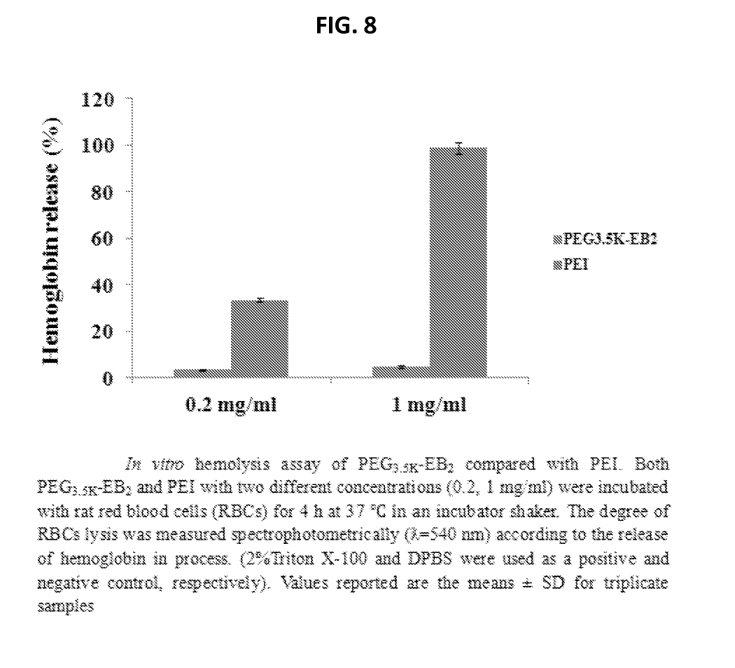

One of the safety concerns for polymeric micelle systems is the hemolytic activity. To address this issue, the hemolytic activity of drug-free PEG3.5K-EB2 micelles was examined and compared to a strong detergent Triton X-100 and polyethylenimine (PEI), a cationic polymer known to have significant hemolytic effect. As shown in FIG. 8, PEI induced hemolysis in a dose-dependent manner. In contrast, no observable hemolytic activities (<5%) were found for PEG3.5K-EB2 micelles, suggesting the excellent safety of our new delivery system.

### In Vitro Cytotoxicity of Paclitaxel Formulated in PEG3.5K-EB2 Micelles:

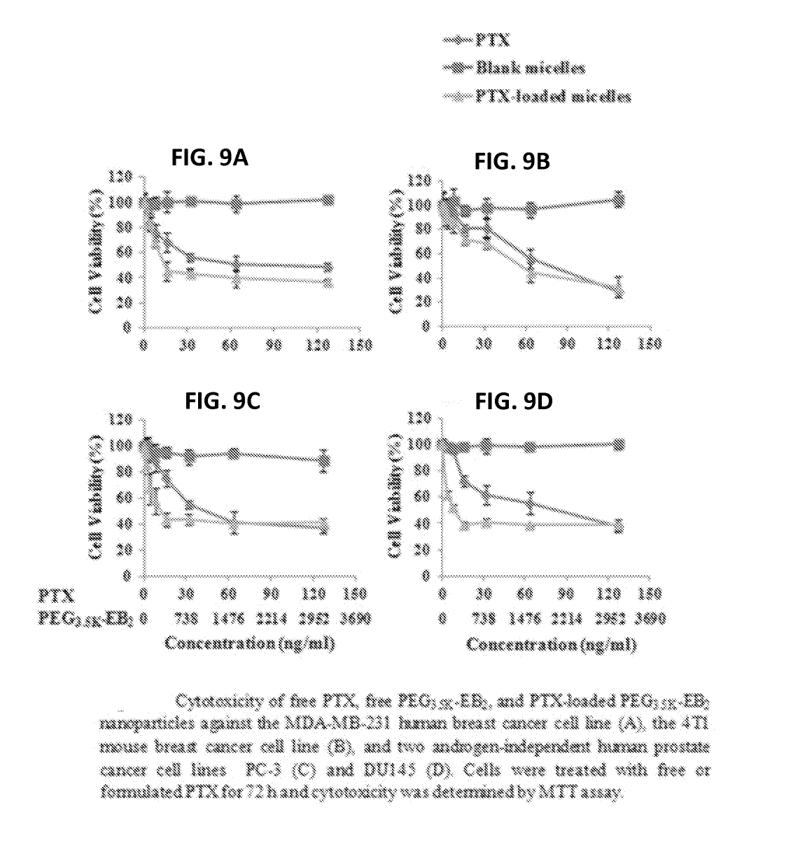

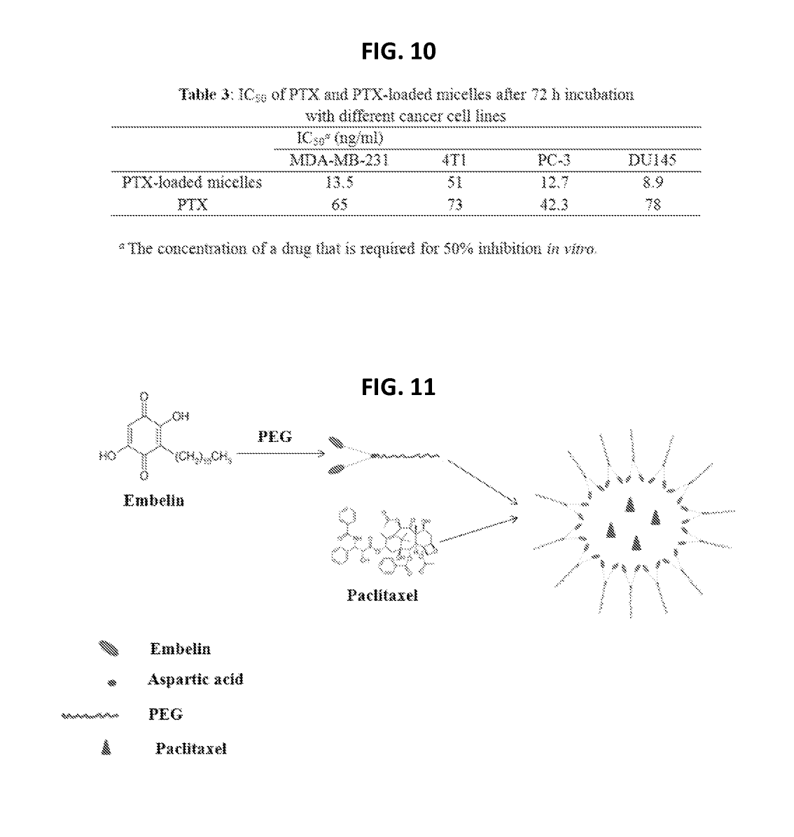

Several cancer cell lines were included in cytotoxicity studies including human breast cancer cells MDA-MB-231, murine breast cancer cells 4T1, and two human prostate cancer cell lines PC3 and DU145. Cells were treated with various concentrations of PTX, free or formulated in PEG3.5K-EB2 micelles, and cytotoxicity was determined by MTT 72 h later. As shown in FIG. 9A, drug-free micelles did not cause any cytotoxicity to MDA-MB-231 cells at the concentration of 3000 ng/mL (the highest concentration used in PTX/PEG3.5K-EB2-treated group). Free PTX exhibited cytotoxicity on MDA-MB-231 cells in a dose-dependent manner. However, formulation of PTX in PEG3.5KEB2-treated micelles resulted in a significant increase in the cytotoxicity. Similar results were found with three other cancer cell lines (FIG. 9B-D). FIG. 10 (Table 3) summarizes the IC50 of free PTX and PEG3.5K-EB2-formulated PTX in the four different cancer cell lines. Dependent on the cell lines, the IC50 was decreased by 1.5- to 8.7-fold when PTX was delivered by PEG3.5K-EB2 micelles.

### Mice Model:

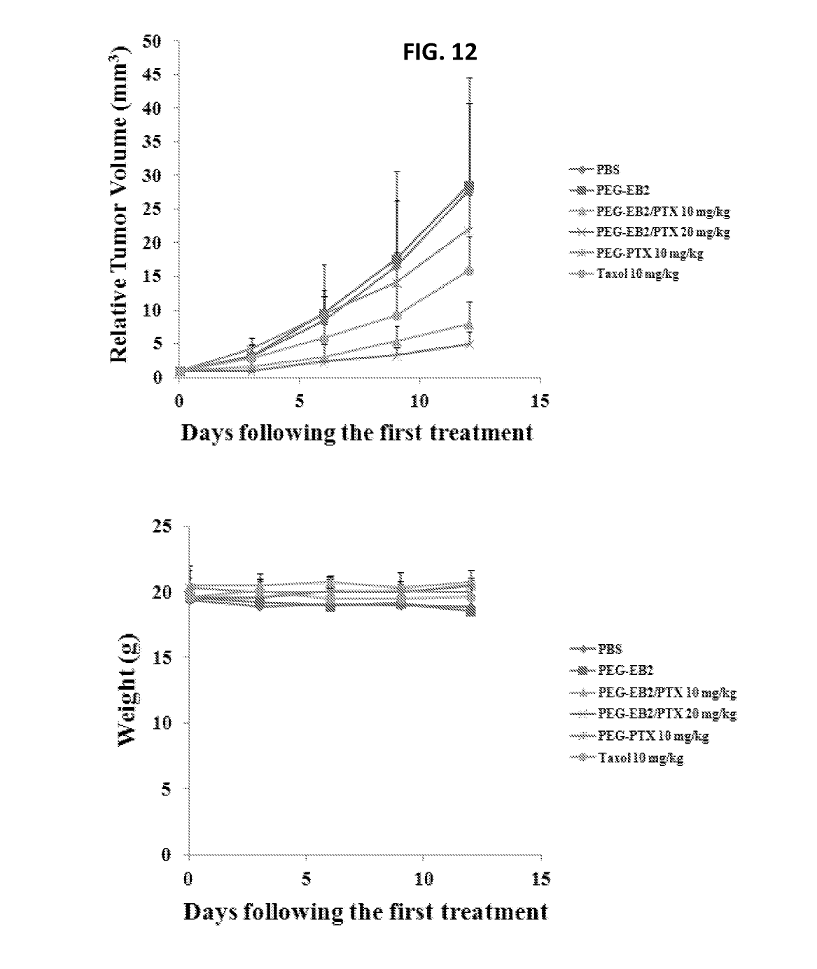

A subcutaneous syngeneic model of breast cancer was established by injecting 1×10⁵ 4 TI cells in a 200 μL PBS subcutaneously at the right flank in female Balb/C mice. Treatments were started when tumor in the mice reached a tumor volume of 50-100 mm³ and this day was designated as day 0. On day 0, these mice were randomly divided into six groups (n=5). Mice were administered intravenously with PBS, free PEG-EB₂ micelles (1337 mg/kg), and PTX-loaded PEG-EB₂ (10 and 20 mg PTX/kg), PEG-PTX (10 mg PTX/kg), Taxol (10 mg/kg) on days 0, 3, 6, 9, 12. Tumor sizes were measured with digital caliper twice a week. The formula used for the tumor calculation is (L×W²)/2, where L is the longest, and W is the shortest in tumor diameters (mm). To compare between groups, relative tumor volume (RTV) was calculated at each measurement timepoint (where RTV equals the tumor volume at given timepoint divided by the tumor volume prior to initial treatment). P<0.005 (PEG-EB₂/PTX vs PBS); P<0.05 (PEG-EB₂/PTX vs PEG-PTX); P<0.05 (PEG-EB₂/PTX vs Taxol). The results are shown in FIG. 12.

The results demonstrate that the PEG_{3.5K}-EB₂ conjugates readily form micelles in aqueous solutions. More importantly, hydrophobic drugs such as PTX can be loaded into PEG_{3.5K}-EB₂ micelles and effectively delivered into cancer cells. Embelin is a natural product that demonstrates various biological effects including antitumor activity.

Embelin also shows excellent safety profiles in animals. Thus, PEG-derivatized embelin may be an attractive delivery system to achieve synergistic activity with anticancer agents while minimizing the carrier-associated toxicity. PEG-embelin conjugates can be synthesized via direct coupling of embelin to PEG via an ester linkage. However, such synthesis is likely to yield a mixture of products with PEG randomly linked to different hydroxyl group in the benzene ring. We have developed a strategy to generate PEG-embelin conjugates via total synthesis (FIG. 1, Scheme 1). This synthesis ensures generation of structurally well-defined conjugates in which PEG is attached to 1-OH group in the quinone ring. All steps give quantitative yields and the synthesis of PEG-embelin conjugates involves similar number of steps and cost as that of embelin alone.

PEG3.5K-EB2 conjugates form small-sized micelles (20˜30 nm) and loading of PTX or embelin did not significantly affect the size of the micelles. The small size of our new micelle system suggests its potential for effective tumor targeting in vivo.

In vitro cytotoxicity with several cancer cell lines showed that PTX formulated in PEG3.5K-EB2 micelles was more active than free PTX in antitumor activity. This is somewhat different from most reported studies in which the micelle-formulated PTX was comparable or less active than Taxol in vitro. This might be consistent with the notion that embelin can sensitize the tumor cells to other anticancer agents. Embelin is coupled to PEG via a cleavable ester linkage. It is likely that embelin is readily released from the conjugates following intracellular delivery and synergizes with PTX in antitumor activity. The carrier itself is not active because embelin alone is active at μM concentrations and the embelin concentrations in the carrier were much lower at the carrier dosages used. However, embelin synergizes with other anticancer agents at subeffective doses.

### PEG_{5K}-Embelin₂

### 2. Experimental Section

### 2.1. Materials

Paclitaxel (98%) was purchased from AK Scientific Inc. (CA, USA). 2,5-dihydroxy-3-undecyl-1,4-benzoquinone (embelin 98%) was purchased from 3B Scientific Corporation (IL, USA). Dulbecco's phosphate buffered saline (DPBS) was purchased from Lonza (MD, USA). Methoxy-PEG_{5,000}-OH, dimethyl sulfoxide (DMSO), 3-(4,5-dimethylthiazol-2-yl)-2,5-diphenyl tetrazolium bromide (MTT), trypsin-EDTA solution, Triton X-100, and Dulbecco's Modified Eagle's Medium (DMEM) were all purchased from Sigma-Aldrich (MO, USA). Fetal bovine serum (FBS), penicillin-streptomycin solution, and DiD (1,1′-dioctadecyl-3,3,3′,3′-tetramethylindodicarbocyanine perchlorate, D-307) were from Invitrogen (NY, USA). All solvents used in this study were HPLC grade.

### 2.2. Synthesis of PEG_{5K}-EB₂

PEG₅ₖ-EB₂ was similarly synthesized according to the method for PEG₃₅ₖ-EB₂. This involves the synthesis of benzoquinone followed by coupling to Boc-aspartic acid. Undecyl side chains were then installed onto each of the two benzoquinone rings. Finally, PEG was coupled to aspartic acid-EB₂ through the deprotected amino group. The final product was analyzed by ¹NMR and MALDI-TOF.

### 2.3. Preparation and Characterization of PTX- and DiD-Loaded PEG_{5K}-EB₂ Micelles

PTX-solubilized micelles were prepared by the following method. PTX (10 mM in chloroform) was added to PEG_{5K}-EB₂ (10 mM in chloroform) with various carrier/drug ratios. The organic solvent was first removed by nitrogen flow to form a thin dry film of drug/carrier mixture. The film was further dried under high vacuum for 2 h to remove any traces of remaining solvent. Drug-loaded micelles were formed by suspending the film in DPBS. The drug-free micelles and DiD-loaded micelles were similarly prepared as described above. The mean diameter of PEG_{5K}-EB₂ micelles with or without loaded drug was assessed by dynamic light scattering (DLS). The morphology and size distribution of drug-free or PTX-loaded PEG₅-EB₂ micelles were observed using transmission electron microscopy (TEM) after negative staining. The CMC of PEG_{5K}-EB₂ was determined by employing pyrene as a fluorescence probe as described above. The concentration of PTX loaded in PEG_{5K}-EB₂ micelles was evaluated by HPLC as described above. The drug loading capacity (DLC) and drug loading efficiency (DLE) were calculated according to the following formula:

DLC (%)=[weight of drug used/(weight of polymer+drug used)]×100%

DLE (%)=(weight of loaded drug/weight of input drug)×100%

### 2.4. In Vitro Drug Release Study

An in vitro drug release study was carried out by dialysis using DPBS (PH=7.4) containing 0.5% (w/v) Tween 80 as the release medium. Taxol formulation was employed as a control. Two mL of PTX-loaded PEG_{5K}-EB₂ micelles or Taxol (1 mg PTX/mL) were sealed in dialysis tubes (MWCO=12 KDa, Spectrum Laboratories) which were then immersed in 200 mL release medium in a beaker covered with parafilm. The beakers were placed in an incubator shaker at 100 rpm and 37° C. The concentration of PTX remaining in the dialysis tubes at various time points was measured by HPLC with the detector set at 227 nm. Values were reported as the means from triplicate samples.

### 2.5. Cell Culture

DU145 and PC-3 are two androgen-independent human prostate cancer cell lines. 4T1.2 is a mouse metastatic breast cancer cell line. All cell lines were cultured in DMEM containing 10% FBS and 1% penicillin-streptomycin in a humidified environment at 37° C. with 5% CO₂.

### 2.6. Cellular Uptake of Nile Red-Loaded PEG_{5K}-EB₂ Micelles

The cellular uptake study was conducted with Nile red as a hydrophobic fluorescence probe. Nile red-loaded PEG_{5K}-EB₂ micelles (7.5:1, m/m, PEG5K-EB2:Nile red) were prepared via a solvent evaporation method as described above. PC-3 cells were seeded in 24-well plates at 2×10⁴ cells per well in 1 mL complete DMEM and cultured for 24 h, followed by removal of culture medium and addition of Nile red-loaded PEG_{5K}-EB₂ micelles at the Nile red concentration of 1 μg/mL. The cells were incubated at 37° C. with 5% CO₂ for 2 h. Subsequently, the nuclei of cells were stained with Hoechst33342 for 5 min. Cells were then washed with DPBS three times and fixed with 4% paraformaldehyde for 30 min at room temperature Finally, the slides were rinsed with DPBS three times and mounted with cover slips and observed under a fluorescence microscope (Eclipse TE300 Microscope).

### 2.7. In Vitro Cytotoxicity Study

The cytotoxicity of PTX formulated in PEG_{5K}-EB₂ micelles was assessed with three cancer cell lines (DU145, PC-3, and 4T1.2) and compared to Taxol formulation. Briefly, DU145, PC-3 or 4T1.2 cells were seeded in 96-well plates followed by 24 h of incubation in DMEM with 10% FBS and 1% streptomycin-penicillin Various dilutions of PTX-loaded PEG_{5K}-EB₂ and Taxol (at the equivalent concentrations of PTX) were added to cells. Controls include PEG_{5K}-EB₂ and Cremophor/ethanol and they were added to cells at concentrations equivalent to those of carriers in the corresponding PTX formulation groups. Cells were incubated for 72 h and cell viability was assessed by MTT assay as described above.

### 2.8. Hemolytic Effect of PEG_{5K}-EB₂ Micelles

Fresh blood samples were collected through cardiac puncture from rats. EDTA-Na₂ was immediately added into 10 mL of blood to prevent coagulation. Red blood cells (RBCs) were separated from plasma by centrifugation at 1500 rpm for 10 min at 4° C. The RBCs were washed three times with 30 mL ice-cold DPBS. RBCs were then diluted to 2% w/v with ice-cold DPBS and utilized immediately for the hemolysis assay. One mL of diluted RBC suspension was treated with various concentrations (0.2 and 1.0 mg/mL) of PEG₅ₖ-EB₂ and PEI, respectively, and then incubated at 37° C. in an incubator shaker for 4 h. The samples were centrifuged at 1500 rpm for 10 min at 4° C., and 100 μL of supernatant from each sample was transferred into a 96-well plate. The release of hemoglobin was determined by the absorbance at 540 nm using a microplate reader. RBCs treated with Triton X-100 (2%) and DPBS were considered as the positive and negative controls, respectively. Hemoglobin release was calculated as (ODₛₐₘₚₗₑ-OD_{negative control})/(OD_{positive control}-OD_{negative control})×100%

### 2.9. Animals

Female BALB/c mice, 10-12 weeks were purchased from Charles River (Davis, Calif.). Male nude mice, 6-8 weeks ages, were purchased from Harlan (Livermore, Calif.). All animals were housed under pathogen-free conditions according to AAALAC guidelines. All animal-related experiments were performed in full compliance with institutional guidelines and approved by the Animal Use and Care Administrative Advisory Committee at the University of Pittsburgh.

### 2.10. Maximum Tolerated Dose (MTD)

Groups of 4 BALB/c mice were administered intravenously with Taxol (15, 20, 25 mg PTX/kg body weight), or PTX-loaded PEG_{5K}-EB₂ micelles (30, 50, 75, 100, 120 mg PTX/kg body weight), respectively. Changes in body weight and survival of mice were followed daily for two weeks. The MTD was defined as the dose that causes neither mouse death due to the toxicity nor greater than 15% of body weight loss or other remarkable changes in the general appearance within the entire period of the experiments.

### 2.11. Biodistribution of PEG_{5K}-EB₂ Micelles Via NIRF Optical Imaging

The in vivo biodistribution and tumor targeting efficiency of PEG_{5K}-EB₂ micelles were investigated by using a near infrared fluorescence dye, DiD. Two nude mice bearing bilateral s.c. PC-3 xenografts were used in this study. Two-hundred μL of DiD-loaded PEG_{5K}-EB₂ micelles were i.v. injected into each mouse and the concentration of DiD in the formulation was 0.4 mg/mL. At indicated times, the two mice were scanned using a Carestream Molecular Imaging System, In-Vivo Multispectral FX PRO, with the excitation at 630 nm and the emission at 700 nm using a 30 second exposure time. Prior to and during each imaging, the mice were anesthetized by isoflurane inhalation. X-ray images were also taken for tumor location and overlaid with corresponding NIR images. After imaging, the mice were euthanized by CO₂ overdose.

### 2.12. In Vivo Therapeutic Study

Two mouse tumor models were used to examine the therapeutic effect of PTX formulated in PEG_{5K}-EB₂ micelles: a syngeneic murine breast cancer model (4T1.2) and a human prostate cancer (PC-3) xenograft model.
For the breast cancer model, 2×10⁵ 4 T1.2 cells in 200 μL PBS were inoculated s.c. at the right flank of female BALB/c mice. Treatments were initiated when tumors in the mice reached a tumor volume around 50 mm³ and this day was designated as day 1. On day 1, mice were randomly divided into six groups (n=5) and received i.v. administration of free PEG_{5K}-EB₂ micelles, Taxol (10 mg PTX/kg), PTX-loaded PEG_{5K}-EB₂, and saline, respectively on days 1, 4, 7, 10, and 13. PTX-loaded PEG_{5K}-EB₂ micelles were given at two different dosages, 10 mg/kg and 20 mg PTX/kg, respectively. Free PEG_{5K}-EB₂ micelles were given at the equivalent dosage of the carrier in the group of PTX-loaded PEG_{5K}-EB₂ micelles (20 mg PTX/kg). Tumor sizes were measured with digital caliper twice a week and calculated according to the following formula: (L×W²)/2, where L is the longest and W is the shortest in tumor diameters (mm). To compare between groups, relative tumor volume (RTV) was calculated at each measurement time point (where RTV equals the tumor volume at a given time point divided by the tumor volume prior to first treatment). Mice were sacrificed when tumor reached 2000 mm³ or developed ulceration.
To monitor the potential toxicity, the body weights of all mice from different groups were measured every three days. In addition, serum level of transaminases (AST, ALT) in the mice treated with PTX/PEG_{5K}-EB₂ (20 mg PTX/kg) and PBS groups was investigated at the completion of the study.
For establishment of PC-3 xenograft tumor model, 2×10⁶ PC-3 cells in 200 μL PBS were inoculated s.c. at the right flank in male nude mice. Treatments were started when tumors in the mice reached a volume around 50 mm³ and different groups (n=6) were similarly treated as described above on days 1, 3, 7, 10, 13, 24, and 28. Tumor size and body weight were monitored as described above.

### 2.13. Statistical Analysis

In all statistical analysis, the significance level was set at a probability of P<0.05. All results were reported as the mean±standard error (SEM) unless otherwise indicated. Statistical analysis was performed by Student's t-test for two groups, and one-way ANOVA for multiple groups, followed by Newman-Keuls test if P<0.05.

### Results

### 3.1. Preparation and Characterization of PTX-Loaded PEG_{5K}-EB₂ Micelles

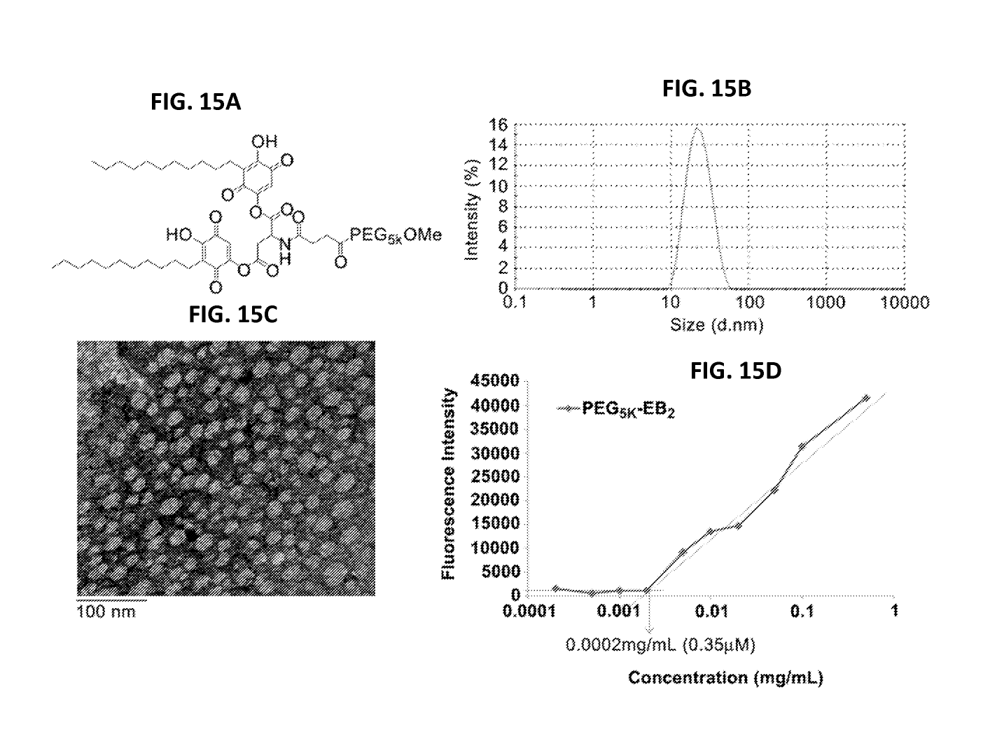

The chemical structure of PEG_{5K}-EB₂ conjugate, in which two embelin molecules were linked to one molecule of PEG_{5K} through a bridge of aspartic acid, is shown in FIG. 15A. The PEG_{5K}-EB₂ conjugate was synthesized via stepwise solution-phase condensation reactions using MeO-PEG-OH, succinic anhydride, Boc-aspartic acid and embelin as building blocks. HPLC shows that the final product (PEG_{5K}-EB₂) is at least 95.57% pure. ¹H NMR spectrum of PEG_{5K}-EB₂ shows signals at 3.63 ppm attributed to the methylene protons of PEG, the embelin proton signals at 8.14 and 6.72 ppm and the carbon chain signals at 1.05-1.25 ppm. The aspartate signals were identified at 5.57, 4.98 and 2.60 ppm. The molecular weight of the PEG_{5K}-EB₂ conjugate from MALDI-TOF MS (5701) is similar to the theoretical value (5703) (FIG. S3), indicating the successful synthesis of PEG_{5K}-EB₂ conjugate.
In aqueous solution, PEG_{5K}-EB₂ readily self-assembles to form micellar nanoparticles with the particle size of around 20 nm as determined by DLS analysis (FIG. 15B). FIG. 15C shows the TEM images of PEG_{5K}-EB₂ micelles after staining with 1% uranyl acetate. Spherical particles of uniform size were observed and the sizes of the micelles observed under TEM were consistent with those measured by DLS.
FIG. 15D shows the CMC of PEG_{5K}-EB₂ micelles using pyrene as a fluorescence probe. Upon incorporation into the micelles, the fluorescence intensity of pyrene increases substantially at the concentration of micelles above the CMC. Based on the partition of the pyrene, the CMC of PEG_{5K}-EB₂ was obtained by plotting the fluorescence intensity versus logarithm concentration of the polymer. The CMC of PEG_{5K}-EB₂ was determined from the crossover point at the low concentration range. The CMC of the PEG_{5K}-EB₂ conjugate is 0.35 μM, which is much lower than most single chain micelle surfactants used in drug delivery systems (mM). The relatively low CMC may render the micelles stable upon dilution in vivo, which is important for effective delivery to tumors.
PEG_{5K}-EB₂ effectively solubilized PTX in aqueous solution. Table 1 compares PEG_{5K}-EB₂ with PEG_{3.5K}-EB₂ with respect to the sizes of PTX-loaded micelles, the drug loading capacity (DLC), and the drug loading efficiency (DLE) under various drug/carrier molar ratios. For PEG_{3.5K}-EB₂ micelles, a minimal 2.5/1 of carrier/PTX molar ratio was required to form stable PTX-loaded micelles. Under this ratio, the size of the drug-loaded micelles was around 143 nm, which was significantly larger than the size of drug-free micelles. Increasing the carrier/PTX ratios resulted in a decrease in the sizes of PTX-formulated micelles. At a carrier/PTX ratio of 7.5/1, the size of PTX-loaded PEG_{3.5K}-EB₂ micelles was similar to that of drug-free micelles.

TABLE 1
Biophysical characterizations of free and drug-loaded PEG-Embelin micelles.

| | | | | Conc. of | | |
|---|---|---|---|---|---|---|
| | Molar | | | PTX in micelles | | |
| Micelles | ratio | Size (nm) | PDI | (%) (mg/ml) | DLC (%) | DLE (%) |
| PEG_{3.5K}-EB₂ | — | 22.8 ± 0.3 | 0.09 | — | — | — |
| PEG_{3.5K}-EB₂:PTX | 2.5:1 | 143 ± 17 | 0.23 | 1 | 7.5 | 79.9 |
| | 5:1 | 58.7 ± 0.5 | 0.32 | 1 | 3.9 | 96.7 |
| | 7.5:1 | 27.5 ± 0.2 | 0.23 | 1 | 2.6 | 98.6 |
| PEG_{5K}-EB₂ | — | 20.6 ± 0.1 | 0.05 | — | — | — |
| PEG_{5K}-EB₂:PTX | 0.75:1 | 25.5 ± 1.0 | 0.06 | 1 | 16.6 | 63.7 |
| | 1:1 | 21.7 ± 0.4 | 0.25 | 1 | 13.0 | 70.8 |
| | | | | 2 | 13.0 | 62.4 |
| | 2.5:1 | 22.0 ± 0.28 | 0.04 | 1 | 5.6 | 93.1 |
| | | | | 2 | 5.6 | 90.8 |
| | 5:1 | 21.9 ± 0.32 | 0.01 | 1 | 2.9 | 98.6 |
| | | | | 2 | 2.9 | 94.9 |
| | 7.5:1 | 22.2 ± 0.14 | 0.11 | 1 | 2.0 | 96.6 |
| | | | | 2 | 2.0 | 94.9 |
| | | | | 3 | 2.0 | 84.4 |

PDI = polydispersity index. DLC = drug loading capacity. DLE = drug loading efficiency. PEG_{3.5K}-EB₂ = PEG_{3.5K}-EB₂. PEG_{5K}-EB₂ = PEG_{5K}-EB₂. PTX = paclitaxel.
PTX concentrations in micelles were kept at 1 mg/mL.
Blank micelle concentration was 20 mg/mL.
Values reported are the means ± SD for triplicate samples.

Compared to PEG_{3.5K}-EB₂, PEG_{5K}-EB₂ conjugate requires much lower carrier/PTX ratios to form stable and small-sized PTX-loaded micelles. PTX-loaded PEG_{5K}-EB₂ micelles still maintained the small size (25 nm) even at the carrier/PTX ratio of 0.75:1 and PTX concentration of 1 mg/mL. Further increase in carrier/drug ratios was associated with an increase in the drug loading efficiency and the PTX concentrations at which PTX-loaded PEG_{5K}-EB₂ micelles remained stable. The improved stability and loading capacity for PEG_{5K}-EB₂ micelles compared to PEG_{3.5K}-EB₂ micelles is likely due to longer PEG brushes capable of providing better steric hindrance and stabilizing effect for micelle nanoparticles.
The size of drug carriers plays a key role in effective targeted delivery to tumors. It has been long known that particles in the size range of 100-200 nm can effectively penetrate solid tumors via an EPR effect. However, a recent study reported that particles with a size of 154 nm were significantly taken up by liver and lungs with limited accumulation at tumor sites [Luo et al., Bioconjug Chem 2010; 21:1216-24]. In contrast, particles with respective size of 17 and 64 nm were much more effective in passive targeting to the solid tumor in a subcutaneous model of human ovarian cancer xenograft [Luo et al.]. The small size of PEG_{5K}-EB₂ micelles (20˜30 nm) may explain their effective in vivo targeting as discussed later.

### 3.2. Release Kinetics of PTX-Loaded Micelles

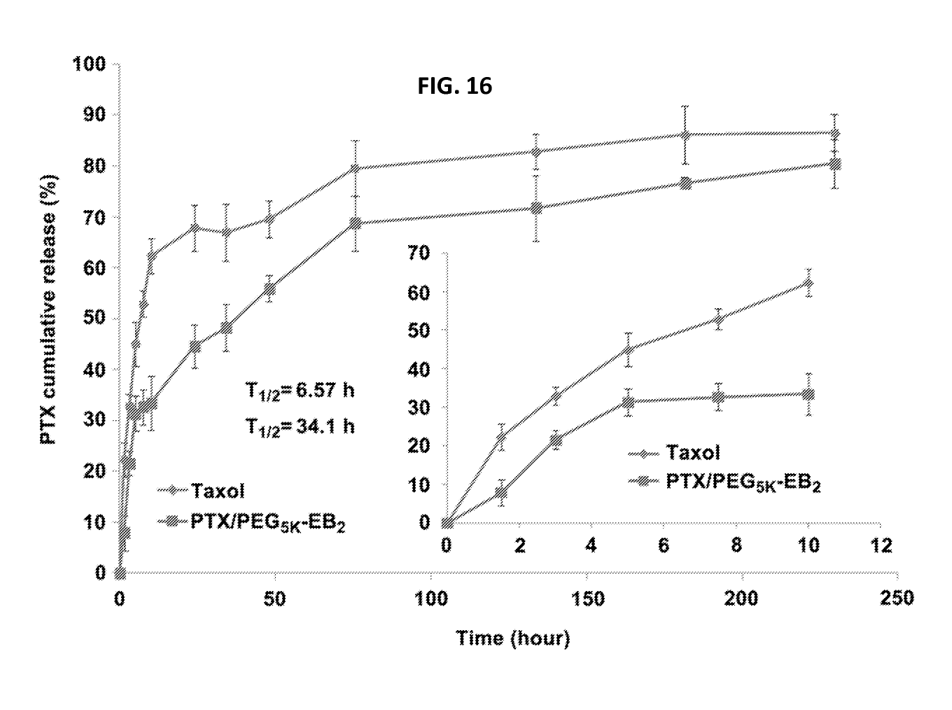

A dialysis method was used to assess the kinetics of release of PTX from PEG_{5K}-EB₂ micelles with DPBS (pH=7.4) containing 0.5% Tween 80 (w/v) as the release medium. Taxol, a clinically used PTX formulation was included as a control. As shown in FIG. 16, PTX formulated PEG_{5K}-EB₂ exhibited significantly better stability than Taxol formulation. For the first 10 h, there was only 33.42% of PTX released from the PEG_{5K}-EB₂ micellar formulation in comparison to the 62.32% release in Taxol formulation. PTX-loaded PEG_{5K}-EB₂ micellar formulation displayed a much slower PTX release compared to Taxol formulation during the entire experimental period. The T_{1/2} of PTX release is 34.1 h for PEG_{5K}-EB₂ micelles, which is significantly longer than that for Taxol formulation (6.57 h). The relatively slower and sustained release in PTX-loaded PEG_{5K}-EB₂ micelle formulation may be ascribed to the strong interaction between the carriers and PTX. Embelin has a benzoquinone ring and a long alkyl chain. In addition to hydrophobic interaction with PTX, the π-π stacking and the hydrogen bonding also contribute to the overall carrier/PTX interaction. The close proximity of two embelins in PEG_{5K}-EB₂ conjugate is likely to facilitate the interaction of the carrier with PTX. Indeed, PEG-embelin conjugates of 1:1 molar ratio were much weaker solubilizer for hydrophobic drugs including PTX (data not shown).

### 3.3. Hemolysis Assay

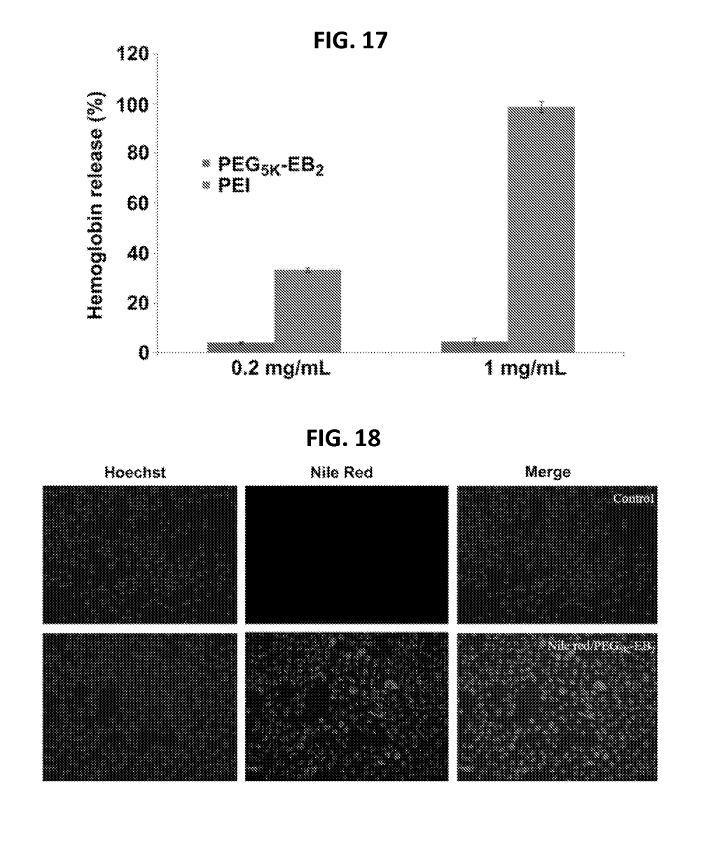

A major concern for micelle systems is whether or not the surface activity of the surfactant molecules affects cell membrane integrity. Therefore, we examined the hemolytic activity of drug-free PEG_{5K}-EB₂ micelles and compared to polyethylenimine (PEI), a cationic polymer with potent cell surface activity. As shown in FIG. 17, treatment of RBCs with PEI resulted in significant hemolysis in a dose-dependent manner. In contrast, no significant hemolysis was observed for blank PEG_{5K}-EB₂ micelles. The negligible hemolytic activity suggests that PEG_{5K}-EB₂ conjugate is a mild surfactant that is suitable for in vivo drug delivery.

### 3.4. Cellular Uptake Study

The cellular uptake of Nile red-loaded PEG_{5K}-EB₂ micelles in prostate cancer cell line PC-3 was investigated by fluorescence microscopy. PC-3 cells were cultured with Nile red-loaded PEG_{5K}-EB₂ micelles (equivalent concentration of Nile red at 1 μg/mL) at 37° C. for 2 h. The nucleus was then stained with Hochest 33342 for 5 mins prior to observation under a fluorescence microscope. As shown in FIG. 18, fluorescence was observed both on the cell membrane and inside the cells with most of the signals located intracellularly. Both perinuclear punctuate and diffuse distribution was observed, suggesting that Nile red-loaded PEG_{5K}-EB₂ was largely taken up by cells via endocytosis and partially released into cytoplasm. Escape of the delivered cargos from endosome into cytoplasm is important as this is where the drug target(s) is located. Although more studies are needed to understand the intracellular trafficking and the underlying mechanism, our data did suggest that PEG_{5K}-EB₂ micelles were capable of effectively mediating intracellular delivery of formulated drugs.

### 3.5. In Vitro Cytotoxicity of PTX-Loaded PEG_{5K}-EB₂ Micelles

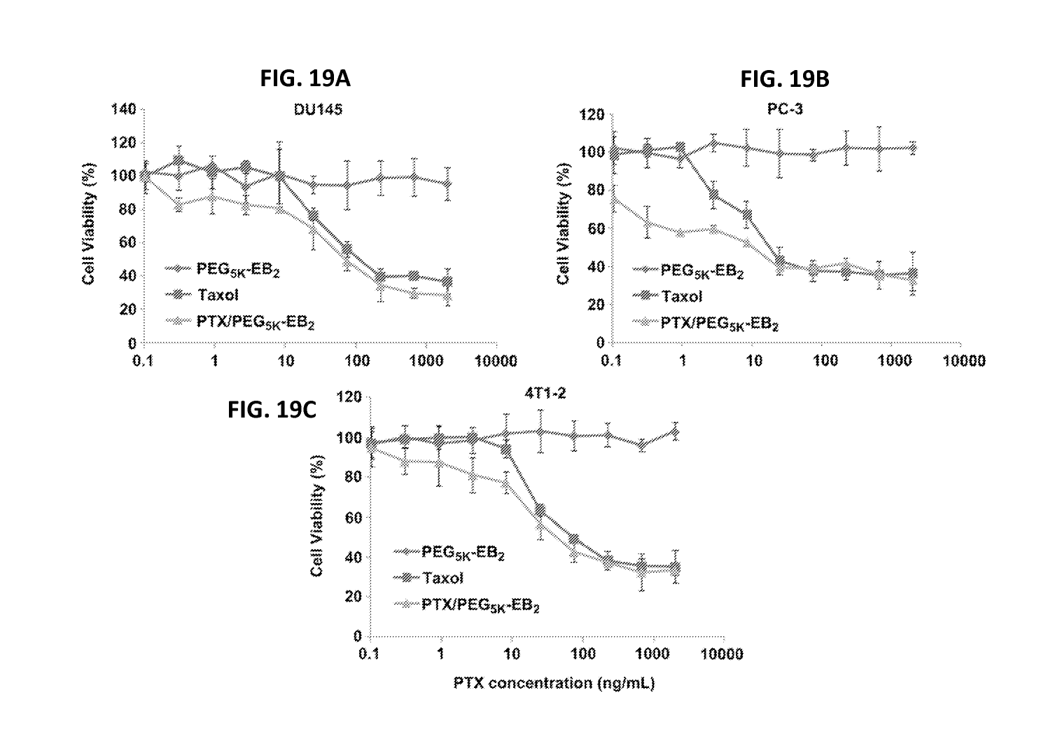

In vitro cytotoxicity of PTX formulated in PEG_{5K}-EB₂ micelles was examined with three cancer cell lines (DU145, PC-3, and 4T1.2) and compared to Taxol formulation. PEG_{5K}-EB₂ alone showed minimal cytotoxic effect to human prostate cancer cells DU145 at the concentrations used to deliver PTX (FIG. 19A). It is also apparent from FIG. 19A that PTX formulated in PEG_{5K}-EB₂ micelles showed higher levels of cytotoxicity to DU145 cells compared to Taxol formulation, particularly at low PTX concentrations. Similar results were obtained in PC-3 (FIG. 19B) and 4T1.2 (FIG. 19C) tumor cells. Most of the reported PTX micellar formulations showed lower or similar levels of cytotoxicity compared to Taxol. The improved in vitro cytotoxicity of PTX formulated in PEG_{5K}-EB₂ micelles may be due to the improved bioavailability of PTX inside the tumor cells. Embelin is coupled to PEG via a cleavable ester linkage, embelin may be freed from the conjugate following intracellular delivery and synergizes with co-delivered PTX in antitumor activity. It should be noted that PEG_{5K}-EB₂ itself is less active in antitumor activity than PEG_{3.5K}-EB₂. This might be due to less effective release of embelin from PEG_{5K}-EB₂ due to a more pronounced steric hindrance imposed by PEG_{5K}.

### 3.6. Maximum Tolerated Dose Study

The maximum tolerated dose for a single i.v. administration of PTX-loaded PEG_{5K}-EB₂ micelles was assessed in tumor-free mice and compared to Taxol. The mice were injected i.v. with different doses of PTX-loaded PEG_{5K}-EB₂ or Taxol followed by daily body weight measurement and observation of general signs of toxicity. As shown in Table 2, Taxol was well tolerated at the dose of 15 mg PTX/kg. However, increasing the PTX dosage to 20 mg/kg resulted in the death of 2 mice among the 4 treated mice. For the mice treated with PTX-loaded PEG_{5K}-EB₂ micelles, there were only 8.7% weight loss and no noticeable changes in normal activity at a PTX dosage as high as 100 mg/kg. At the dosage of 120 mg PTX/kg, two out of 4 treated mice died of toxicity. Based on these data it was estimated that the single i.v. MTD for Taxol was 15˜20 mg PTX/kg while that for PTX-loaded PEG_{5K}-EB₂ micelles was 100˜120 mg PTX/kg. The MTD for PTX-loaded PEG_{5K}-EB₂ micelles is higher than most of the reported PTX formulations. The high MTD for PTX/PEG_{5K}-EB₂ is likely due to the slow release kinetics for PTX (FIG. 16), low levels of nonselective uptake by major organs (see later), and the excellent safety profile of embelin. Embelin has antiinflammatory and hepatoprotective activity. In addition, normal tissues are less sensitive to embelin compared to tumor cells due to the significantly lower levels of XIAP expression in normal tissues. The significantly improved safety of our delivery system over Taxol formulation will allow high dosage of PTX to be given to achieve maximal therapeutic effect.

TABLE 2
Animal deaths and weight loss in the MTD study.

| | Does | Animal | Weight |
|---|---|---|---|
| Formulations | (mg/kg) | death | loss (%) |
| Taxol | 15 | 0/4 | 5.4 |
| | 20 | 2/4 | N/A |
| | 25 | 4/4 | N/A |
| PTX-loaded | 30 | 0/4 | −1.2 |
| PEG_{5K}-EB₂ micelles | 50 | 0/4 | 1.6 |
| | 75 | 0/4 | 6.5 |
| | 100 | 0/4 | 8.7 |
| | 120 | 2/4 | N/A |

### 3.6. Maximum Tolerated Dose Study

The maximum tolerated dose for a single i.v. administration of PTX-loaded PEG_{5K}-EB₂ micelles was assessed in tumor-free mice and compared to Taxol. The mice were injected i.v. with different doses of PTX-loaded PEG_{5K}-EB₂ or Taxol followed by daily body weight measurement and observation of general signs of toxicity. As shown in Table 2, Taxol was well tolerated at the dose of 15 mg

### 3.7. Biodistribution of PEG_{5K}-EB₂ Micelles Via NIRF Optical Imaging

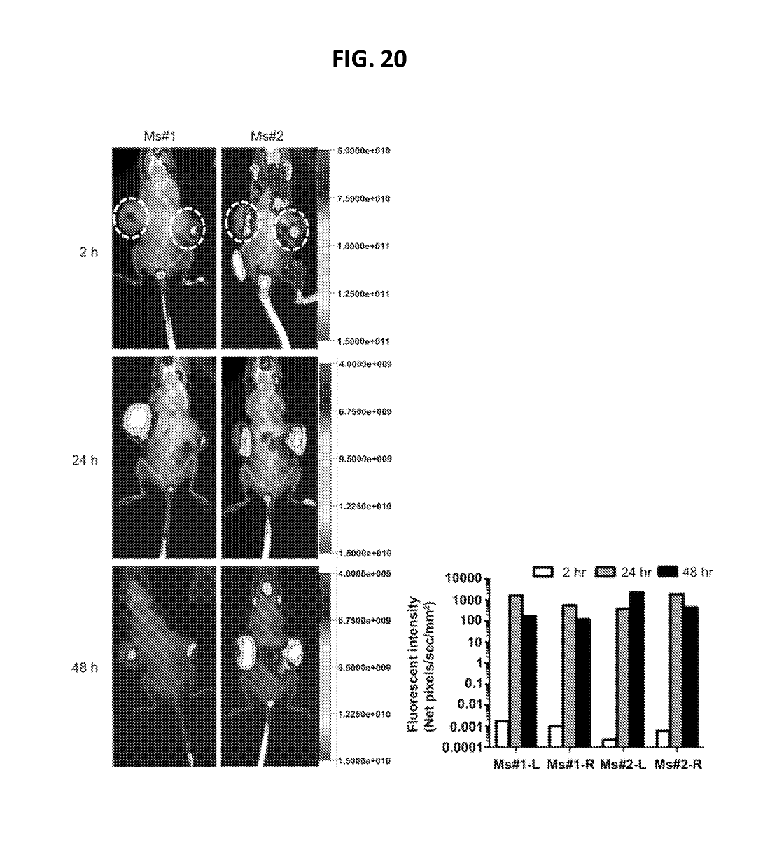

Biodistribution and tumor targeting efficiency of PEG_{5K}-EB₂ micelles were evaluated in a mouse xenograft model of human prostate cancer (PC-3), using a hydrophobic near infrared fluorescence (NIRF) dye, DiD. Two hundred μL of micelles co-loaded with PTX and DiD was intravenously injected into two mice bearing bilateral PC-3 tumors, respectively. The two mice were then followed over time by the scanning with Carestream Molecular Imaging System. FIG. 20 shows the imaging of the tumor-bearing mice at 2, 24, 48 h following i.v. injection of PTX/PEG_{5K}-EB₂ mixed micelles carrying DiD. A noticeable signal in tumor was observed as early as 2 h post injection; the signal peaked around 24 h and remained clearly visible 48 h after injection. Interestingly, little fluorescence signal was observed in liver and spleen, the two major internal organs that are involved in the nonspecific clearance of nanoparticles by the reticuloendothelial system (RES). The effective targeting of PEG_{5K}-EB₂ micelles to the tumors and the minimal uptake by RES system are largely due to the very small-sized particles, excellent PEG shielding effect, and a likely excellent stability in the blood circulation.

### 3.8 In Vivo Therapeutic Study

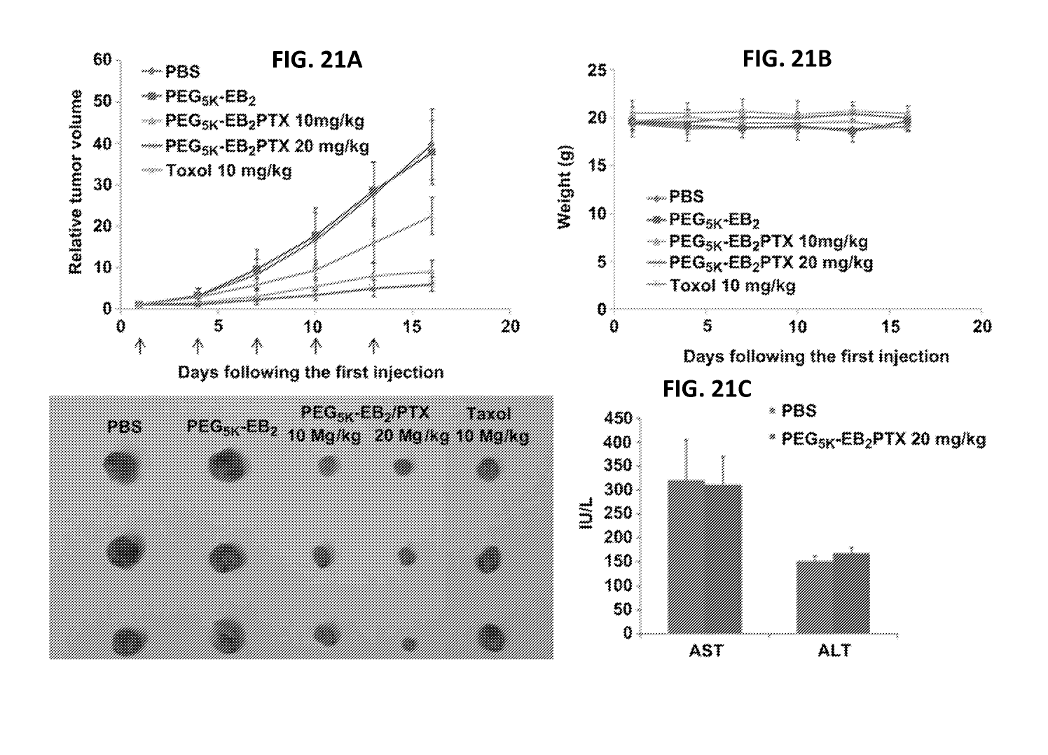

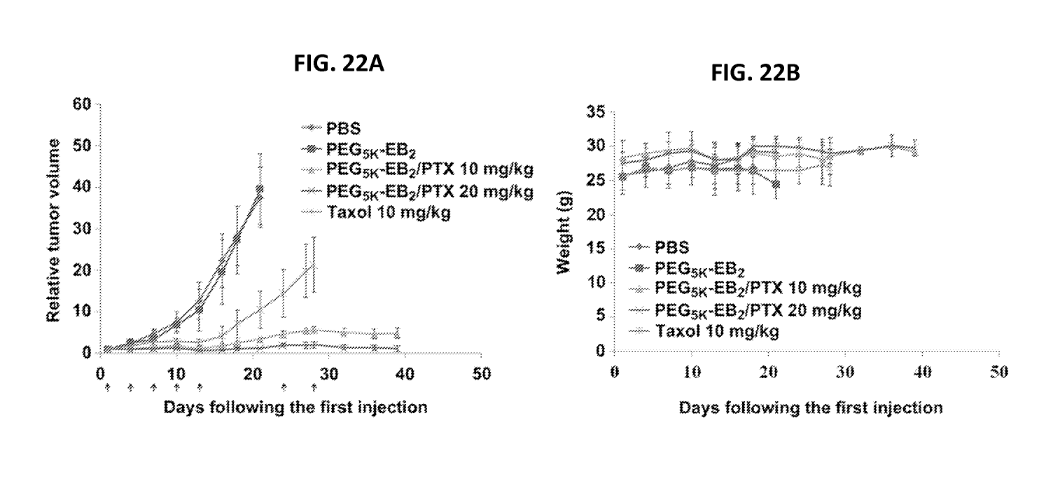

The in vivo therapeutic activity of PTX formulated in PEG_{5K}-EB₂ micelles was investigated in two mouse tumor models: a syngeneic murine breast cancer model (4T1.2) and a human prostate cancer xenograft model (PC-3).
4T1.2 is a highly metastatic breast cancer cell line and was chosen in this study to stringently assess the therapeutic efficacy of our new delivery system. As shown in FIG. 21A, PEG_{5K}-EB₂ alone showed no effect in inhibiting the tumor growth. This is likely due to a low concentration of embelin in this group. Taxol formulation showed a modest effect in inhibiting the tumor growth at a dose of 10 mg PTX/kg. In contrast, PTX formulated in PEG_{5K}-EB₂ micelles showed a much more pronounced antitumor activity at the same dosage. Increasing the PTX dosage to 20 mg/kg resulted in a further improvement in the therapeutic effect. No significant changes in body weight were noticed in all treatment groups compared to PBS control group (FIG. 21B). In addition, serum levels of transaminases in the mice treated with the high dose of PTX-loaded PEG_{5K}-EB₂ micelles were comparable to those in PBS control group (FIG. 21C), suggesting that significant therapeutic effect can be achieved with minimal toxicity using our new delivery system.
Following the demonstration of effective antitumor activity in the syngeneic murine breast cancer model, the in vivo therapeutic effect of PTX-loaded PEG_{5K}-EB₂ micelles was further investigated in a human prostate cancer xenograft model (PC-3). PC-3 tumor-bearing mice were similarly treated as described in the study with the 4T1.2 tumor model and the data are shown in FIG. 22A. It is apparent that tumor growth was more effectively controlled by PTX/PEG_{5K}-EB₂ micelles in PC-3 model compared to 4T1.2 tumor model. By day 16 after the first treatment, the tumor growth was completely suppressed with a RTV of 0.84 in the group treated with a high dose (20 mg PTX/kg) of PTX-loaded PEG_{5K}-EB₂ micelles. Tumor growth was also significantly slowed in the group treated with a low dose (10 mg PTX/kg), in which the tumors only reached a RTV of 1.75. This compared very favorably to Taxol group, in which RTV reached 4.24. Although the tumors started to recover slightly after day 24 in the two groups treated with PTX/PEG_{5K}-EB₂ mixed micelles, RTV was reduced back to 1.15 at day 39 in the high dose group following two additional treatments at days 24 and 28. In fact, two out of 6 mice in this group became tumor-free after day 32 without further treatment. The growth of tumor in the low dose group also became static after two additional treatments. In contrast, tumors in Taxol group continued to grow at a steady and fast rate. No noticeable changes in weight were shown from direct measurement of tumor-bearing mice in all groups (FIG. 22B). The superior anti-tumor efficacy along with the minimal toxicity of PTX/PEG_{5K}-EB₂ micelles could be ascribed to their high efficiency in tumor-targeting and minimal nonspecific uptake by RES (FIG. 20). The slow release kinetics of PTX/PEG_{5K}-EB₂ micelles may also contribute to the enhanced antitumor activity. A conjugate of PEG_{5K} with two embelin molecules (PEG_{5K}-EB₂) forms small sized micelles (20˜30 nm) that effectively solubilize hydrophobic drugs such as PTX. Compared to a similar conjugate with a lower MW PEG (PEG_{3.5K}-EB₂), PEG_{5K}-EB₂ gives increased drug loading capacity and forms stable drug-loaded micelles at lower carrier/drug ratios. PEG_{5K}-EB₂ micelles have a low CMC and are effective in mediating intracellular delivery of loaded agents. PTX-loaded PEG_{5K}-EB₂ micelles show a kinetics of sustained release and are effectively targeted to tumors in vivo with minimal nonspecific uptake by RES. PTX formulated in PEG_{5K}-EB₂ micelles exhibited potent cytotoxicity to several cultured cancer cell lines. In vivo, PTX-loaded PEG_{5K}-EB₂ micelles demonstrated an excellent safety profile with a MTD of 100˜120 mg PTX/kg, which was significantly higher than that (15˜20 mg PTX/kg) for Taxol. Furthermore, superior antitumor activity over Taxol formulation was demonstrated in both breast cancer and prostate cancer models. In a human prostate cancer xenograft model (PC-3), complete inhibition of tumor growth was achieved with minimal toxicity to the animals.

### DOX-loaded PEG_{5K}-EB₂ Micelles

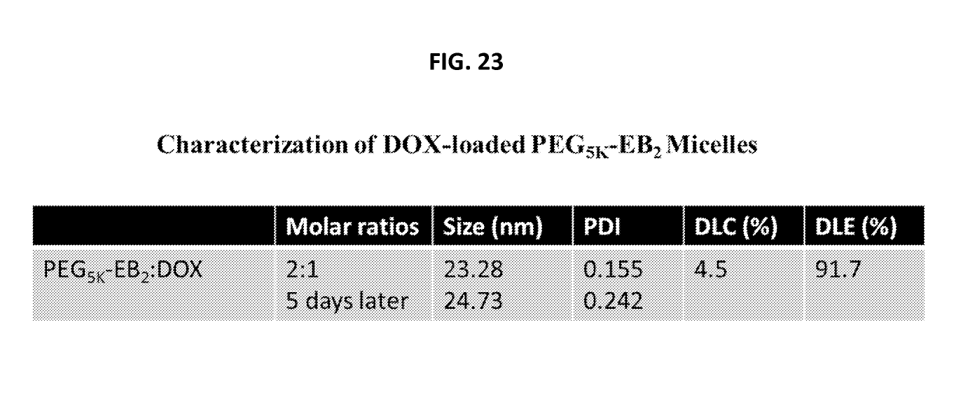

Doxorubicin (DOX) is a partially hydrophobic anticancer drug with known toxic side effects towards cardiovascular systems. Free DOX-HCl salt is water soluble, while its free-base is less soluble and can be efficiently incorporated into the PEG-EB micelles. The DOX-loaded PEG_{5K}-EB₂ has very small particles sizes and good stability over time (see FIG. 23). The presence of PEG-brushes on the micelle surface may minimize the clearance by mononuclear phagocyte systems found in liver and spleen. This, together with the good particle stability of DOX-loaded PEG_{5K}-EB₂ micelles may allow for an extended blood circulation time. Moreover, small-sized micelles promote efficient extravasation and subsequent preferential accumulation in tumor masses due to a enhanced permeability and retention (EPR) effect, resulting in reduced systemic side effects of DOX.

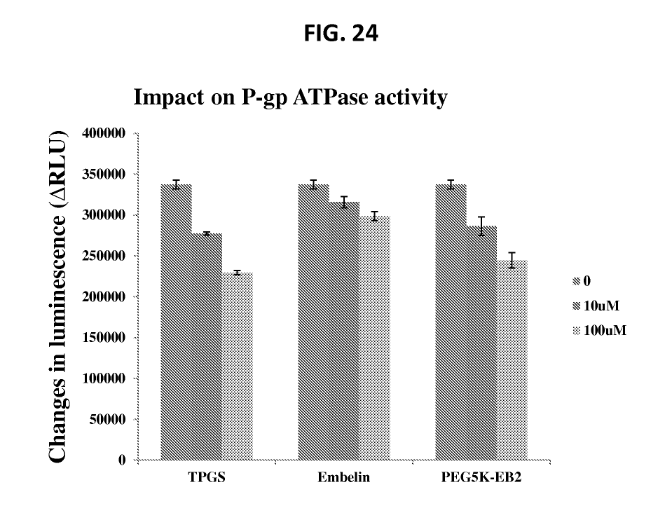

P-glycoprotein 1 (Pgp) or multidrug resistance protein 1 (MDR1) is a membrane transporter that transports a wide variety of substrates across membranes at the expense of ATP. Although the normal function of Pgp is to exclude toxic substances from cells by pumping these out of the cells, cancer cells often overexpress this protein to get rid of multiple types of anti-cancer drugs to gain survival advantages. TPGS is a vitamin E derivative known to block this pump activity. An assay was performed measuring the channel blocking activity of various agents include TPGS, free embelin and PEG-embelin conjugate, on the hydrolysis of ATPase activity for PgP; and the change of ATP levels (measured by the amount of light generation by luciferase) was used as an indicator of the inhibitory activities. The results are shown in FIG. 24. PEG_{5K}-EB₂ showed comparable efficacy realtive to TPGS in inhibiting the Pgp ATPase activity.

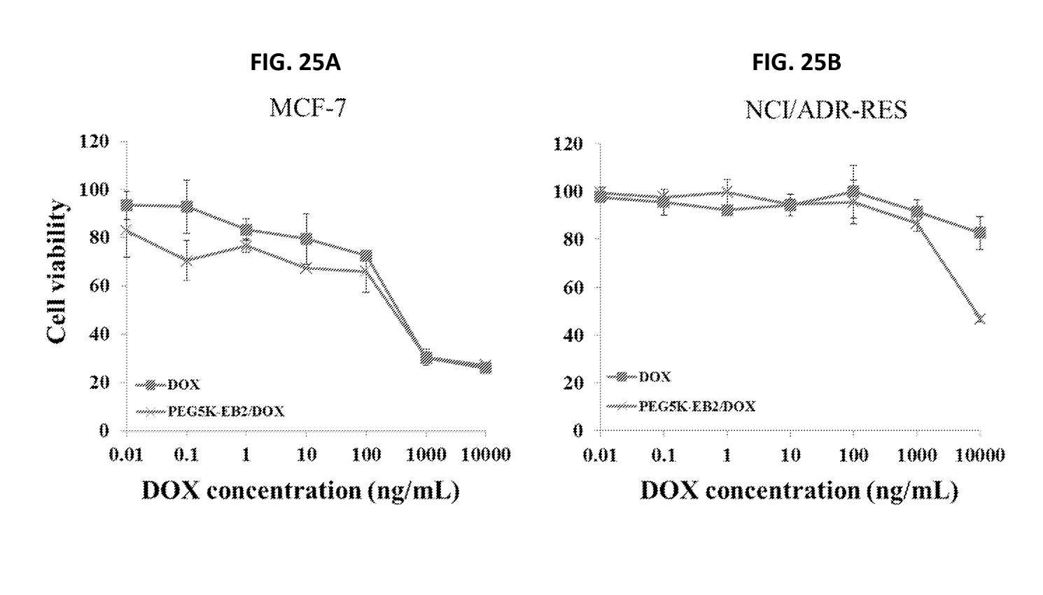

The data shown in FIGS. 25A and 25B shown that PEG-EB conjugate can reverse multiple drug resistance phenotype of cancer cell lines, and that for Pgp overexpressed cancer cell line-NCI/ADR-RES cells, DOX formulated in PEG_{5K}-EB₂ clearly showed better antitumor activity than that of DOX.

In view of the many possible embodiments to which the principles of the disclosed invention may be applied, it should be recognized that the illustrated embodiments are only preferred examples of the invention and should not be taken as limiting the scope of the invention.

1. A composition comprising a structure of:
X-L-Y
wherein X comprises at least one embelin moiety;
L comprises a linker; and
Y comprises a hydrophilic moiety.

2. The composition of claim 1, wherein the embelin moiety comprises embelin or an embelin analog.

3. The composition of claim 2, wherein the embelin moiety comprises a structure of: 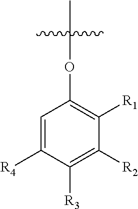
wherein each of R₁, R₃, and R₄ is individually hydroxyl, oxo, or methoxy; and R₂ is a hydrophobic moiety selected from an optionally substituted alkyl having at least 6 carbon atoms, or an optionally substituted aryl, or a tautomer thereof.

4. The composition of claim 1, wherein the linker is derived from an amino acid, a carbonyl-containing molecule, or an ether-containing molecule.

5. The composition of claim 4, wherein the linker comprises an aspartic acid structure.

6. The composition of claim 1, wherein the hydrophilic moiety includes at least one of SO₃⁻², COO⁻¹, PO₄⁻³, (CH₃)₃N⁺¹, (CH₃CH₂)₃N⁺¹, (HOCH₂CH₂)₃N⁺¹, methyl pyridine⁺¹, multivalent cationic group, a polyalkylene glycol, polyglycerol, poly(vinyl alcohol), or a mono, oligo or polysaccharide.

7. The composition of claim 6, wherein the hydrophilic moiety comprises a polyethylene glycol.

8. The composition of claim 1, wherein:
the embelin moiety comprises a structure of: 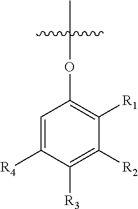
wherein each of R₁, R₃, and R₄ is individually hydroxyl; and R₂ is an alkyl having at least 6 carbon atoms;
the linker comprises an aspartic acid structure; and
the hydrophilic moiety comprises a polyethylene glycol.

9. The composition of claim 1, wherein the composition is formulated as a micelle.

10. The composition of claim 1, further comprising a targeting moiety bonded to the embelin moiety, the linker, or the hydrophilic moiety.

11. The composition of claim 10, wherein the targeting moiety comprises a small molecule ligand for σ2 receptor.

12. The composition of claim 1, wherein the composition is a conjugate.

13. A micelle comprising:
a core that includes at least one hydrophobic active agent and at least one embelin moiety; and
a hydrophilic zone surrounding the core and comprising at least one hydrophilic moiety.

14. The micelle of claim 13, wherein the active agent is a pharmaceutically active agent.

15. The micelle of claim 14, wherein the pharmaceutically active agent is selected from embelin, amphteracin B, doxurobicin, cyclosporine, FK506, a taxane, or camptothecin.

16. The micelle of claim 13, wherein the embelin moiety comprises a structure of: 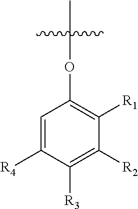
wherein each of R₁, R₃, and R₄ is individually hydroxyl, oxo, or methoxy; and R₂ is a hydrophobic moiety selected from an optionally substituted alkyl having at least 6 carbon atoms, or an optionally substituted aryl, or a tautomer thereof.

17. The micelle of claim 13, wherein the hydrophilic moiety includes at least one of SO₃⁻², COO⁻¹, PO₄⁻³, (CH₃)₃N⁺¹, (CH₃CH₂)₃N⁺¹, (HOCH₂CH₂)₃N⁺¹, methyl pyridine⁺¹, multivalent cationic group, a polyalkylene glycol, polyglycerol, poly(vinyl alcohol), or a mono, oligo or polysaccharide.

18. The micelle of claim 13, wherein the embelin moiety and the hydrophilic moiety are conjugated to each other via a linker.

19. The micelle of claim 18, wherein the linker is derived from an amino acid, a carbonyl-containing molecule, or an ether-containing molecule.

20. The micelle of claim 19, wherein the linker comprises an aspartic acid structure.

21. The micelle of claim 18, further comprising a targeting moiety bonded to the embelin moiety, the linker, or the hydrophilic moiety.

22. A composition comprising the micelle of claim 13 and a continuous phase in which the micelle is solubilized.

23. The composition of claim 22, wherein the continuous phase comprises an aqueous phase.

24. The composition of claim 22, wherein the continuous phase is an aqueous phase.

25. A method for making a micelle-containing composition, comprising mixing the micelle of claim 13 together with an aqueous phase.

26. The method of claim 24, wherein the aqueous phase is selected from water or saline.

27. A method comprising administering a therapeutically effective amount of the composition of claim 1 to a subject in need thereof.

28. A method comprising administering a therapeutically effective amount of the micelle of claim 13 to a subject in need thereof.

29. A method comprising administering a therapeutically effective amount of the composition of claim 22 to a subject in need thereof.

30. The method of claim 27, wherein the method comprises treating cancer in the subject.

31. The method of claim 30, wherein the pharmaceutically active agent is a taxane and the method comprises treating prostate cancer or breast cancer in the subject.

32. The method of claim 30, wherein the cancer is a drug-resistant cancer.

33. The method of claim 30, wherein the pharmaceutically active agent is doxurobicin.
